(19) United States
(12) Patent Application Publication
(10) Pub. No.: US 2021/0228644 A1
(43) Pub. Date: Jul. 29, 2021

(54) NEURAL STEM CELL COMPOSITIONS AND METHODS TO TREAT NEURODEGENERATIVE DISORDERS

(71) Applicant: The Regents of the University of California, Oakland, CA (US)

(72) Inventors: John REIDLING, Irvine (CA); Brian FURY, Davis (CA); Leslie Michels THOMPSON, Irvine (CA); Gerhard Bauer, Davis (CA); Dane COLEAL-BERGUM, Davis (CA)

(73) Assignee: The Regents of the University of California, Oakland, CA (US)

(21) Appl. No.: 16/972,450

(22) PCT Filed: Jun. 6, 2018
(86) PCT No.: PCT/US2018/036355

§ 371 Date: Dec. 4, 2020

(51) Int. Cl. A61K 35/30; C12N 5/0797; A61P 25/28

(52) U.S. Cl. CPC A61K 35/30; C12N 2510/00; A61P 25/28; C12N 5/0623

(57) ABSTRACT

Provided herein are stem-cell based therapies for the treatment of neurodegenerative diseases and CNS disorder such as Huntington's disease. The therapy improved motor deficits and rescued synaptic alterations. The cells were shown to be electrophysiologically active and that they improved motor and late-stage cognitive impairment.

### BACKGROUND

Currently no disease-modifying therapies are available for many neurodegenerative disorders that affect the central or peripheral nervous system. Some have suggested that human stem cells offer a possible therapeutic strategy for some neurodegenerative disorders (for reviews see Drouin-Ouellet, 2014; Golas and Sander, 2016; Kirkeby et al., 2017).

As an example, Huntington's disease (HD) is an autosomal dominant neurodegenerative disease caused by an expanded CAG repeat encoding a polyglutamine repeat within the Huntingtin protein (HTT) (The Huntington's Disease Collaborative Research Group, 1993). Involuntary movements, progressive intellectual decline, and psychiatric disturbances occur (Ross and Tabrizi, 2011), and neuropathology primarily involves degeneration of medium-sized spiny neurons (MSNs) in the striatum and atrophy of the cortex (Vonsattel and DiFiglia, 1998). A need exists in the art to find treatment for neurodegenerative diseases and disorders such as HD. This disclosure satisfies this need and provides related advantages as well.

### SUMMARY

Provided herein is a method to prepare a human neuronal stem cell (hNSC) from a human embryonic stem cell (hESC), the method comprising, or alternatively consisting essentially of, or yet further consisting of, the steps of:
a) isolating at least one stem cell rosette from a population of embryoid bodies (EB) cultured in differentiation medium;
b) culturing at least one individual cell isolated from the rosette of step a) for an amount of time and under until conditions that provide for the generation of at least one rosette;
c) isolating an individual cell from the rosette of step b) into individual cells; and
d) culturing the at least one individual cell isolated from step c) for an amount of time and under until conditions that provide for the generation of confluent population of hNSCs.

In some embodiments, the isolation of the at least one individual cell from the rosette is performed manually. In another aspect, the isolation of the at least one individual cell from the rosette is performed enzymatically. In a further aspect, the isolation of the at least one individual cell from the rosette of step a) is performed digitally, optionally using digital two or three dimensional image recognition technology. In a yet further aspect, the isolation of the at least one individual cell of step c) is performed enzymatically.

In some embodiments, the one or more of steps a) through c) is performed 2 or more times can be performed, manually, or mechanically in a high throughput manner, optionally using digital two or three dimensional image recognition technology.

In some embodiments, the method further comprises generating the embryoid bodies from ESI-017. In some embodiments, the method further comprises culturing the embryoid body (EB) on an ultra-low attachment surface in EB medium. In some embodiments, the method further comprises substituting N2 medium for the EB medium after the EBs have been cultured for an effective amount of time further to step a) on an ornithine/laminin coated surface. In some embodiments, the method further comprises substituting N2 medium for the EB medium after the EB have been cultured in the EB medium for an amount of time effective to produce at least one EB of step a).

In some embodiments, at least one individual cell isolated in step c) is cultured for an effective amount of time on an ornithin/laminin coated plate in N2 medium to generate a confluent cell population of hNSCs. In some embodiments, the method further comprises culturing the confluent population of hNSCs with an effective amount of N2 medium. In some embodiments, the method further comprises expanding the population of cells.

In some embodiments, the method further comprises genetically modifying the cell. In some embodiments, the cell is genetically modified by insertion of a transgene, or by modification by CRISPR. In some embodiments, the transgene is ApiCCT1, a fragment thereof, or an equivalent of each thereof, and optionally wherein the transgene is overexpressed in the cell.

In some aspects, provided herein is an hNSC prepared by comprising, or alternatively consisting essentially of, or yet further consisting of, the steps of: a) isolating at least one stem cell rosette from a population of embryoid bodies (EB) cultured in differentiation medium;

b) culturing at least one individual cell isolated from the rosette of step a) for an amount of time and under until conditions that provide for the generation of at least one rosette;

c) isolating an individual cell from the rosette of step b) into individual cells; and

d) culturing the at least one individual cell isolated from step c) for an amount of time and under until conditions that provide for the generation of confluent population of hNSCs.

In some embodiments, the hNSC expresses BNDF. In some embodiments, the hNSC expresses BNDF upon differentiation of the cell. In some embodiments, the cell is genetically modified by insertion of a transgene, or by CRISPR.

In some aspects, provided herein is a population of cells prepared according to the methods described herein. Also provided are compositions comprising an isolated cell prepared according to the methods described herein. In some embodiments, the composition further comprises a carrier. In some embodiments, the carrier is a preservative and/or cryoprotectant.

In some aspects, provided herein is a method to deliver a transgene to a subject, or to genetically edit a cell in a subject in need thereof, comprising administering an effective amount of an isolated cell prepared according to the methods described herein. In some embodiments, the subject is a mammal. In some embodiments, the subject is a human.

In some aspects, provided herein is a method of treating a neurodegenerative disorder or enhancing synaptic connections in a subject in need thereof, comprising administering an effective amount of an isolated cell prepared according to the methods described herein. In some embodiments, the subject is a mammal. In some embodiments, the subject is a human. In some embodiments, the neurodegenerative disorder is selected from the group of Huntington's disease, stroke, Alzheimer's disease, Parkinson's disease, traumatic brain injury, brain inflammation, stroke, autoimmune disorders such as multiple sclerosis, primary or secondary progressive multiple sclerosis, relapsing remitting multiple sclerosis, chronic spinal cord injury, Bell's palsy, cervical spondylosis, carpal tunnel syndrome, brain or spinal cord tumors, peripheral neuropathy, Guillain-Barre syndrome, spinal muscular atrophy, Freidrich's ataxia, amyotrophic lateral sclerosis, and Huntington chorea.

In some aspects, provided herein are kits comprising an hESC and instructions for performing a method as described herein.

In some aspects, provided herein is a non-human animal having an hNSC prepared according to the methods described herein and transplanted into the animal. In some embodiments, the animal is a murine or ovine.

### BRIEF DESCRIPTION OF THE DRAWINGS

FIGS. 1A-1D: ESI-017 hNSCs Implanted in R6/2 Mice Improve Behavior and Exhibit Evidence of Differentiation into Immature Neurons and Astrocytes. (A) Rotarod task demonstrates a deficit in R6/2 mice compared with non-transgenic littermates (NT), and hNSC-treated R6/2 mice have increased average latency to fall 1 week (black bars) and 3 weeks (gray bars) after implantation compared with vehicle-treated (Veh) mice. (B) Pole test demonstrates a deficit with R6/2 mice compared with NT. hNSC-treated R6/2 mice descend faster than Veh mice 4 weeks after implantation (gray bars) but not 2 weeks after implantation (black bars). (C) Grip strength demonstrates a deficit in R6/2 mice compared with NT. hNSC-treated R6/2 mice have greater grams of strength after 4 weeks compared with Veh mice (black bars) but not after 2 weeks (gray bars). (D) Immunohistochemistry (IHC). hNSCs (human marker SC121) implanted in striatum of R6/2 mice co-localize with marker for neuron-restricted progenitors (doublecortin [DCX], and astrocytes (SC121 and GFAP). One-way ANOVA followed by Tukey's HSD test with Scheffe', Bonferroni, and Holm multiple comparison calculation performed post hoc. *p<0.05, **p<0.01 (n=15). Graphs show means±SEM.

FIGS. 2A-2F: IHC Shows that ESI-017 hNSCs Implanted in R6/2 Mice Differentiate. (A) hNSCs (SC121) implanted in R6/2 mice differentiate into neuron-restricted progenitors (doublecortin [DCX]) and astrocytes (SC121 and GFAP). (B) High magnification (633) showing differentiation: hNSCs (human nuclear marker Ku80) implanted in R6/2 mice differentiate into neuron-restricted progenitors (DCX) and some astrocytes (Ku80 and GFAP). (C) hNSCs (Ku80) and neuron-restricted progenitors (DCX). (D) hNSCs (Ku80) and neuron-restricted progenitors (βIII-tubulin); mouse cell nuclei shown with DAPI. (E) hNSCs (Ku80) and neuron-restricted progenitors (MAP-2); mouse cell nuclei shown with DAPI. (F) hNSCs (Ku80) do not co-localize with differentiated post-mitotic neuronal cell marker (NeuN).

FIGS. 3A-3F: Implantation of ESI-017 hNSCs Reduces Corticostriatal Hyperexcitability in R6/2 Mice. (A) Biocytin-filled (arrow) hNSC that was recorded in the striatum and IHC with SC121. Scale bar, 20 mm. (B) Top trace: cell-attached recording of spontaneously firing hNSC. Bottom traces: sEPSCs and sIPSCs from hNSC. Recordings illustrate spontaneous inward and outward synaptic currents in the hNSC. (C) sEPSCs and sIPSCs recorded in MSN. (D) Biocytin-filled MSN near a cluster of hNSCs (SC121). Scale bar, 20 mm. (E) Recordings of sEPSCs in a subpopulation of R6/2 MSNs show “epileptiform” activity after the addition of the GABAA receptor antagonist, bicuculline (10 mM) (first trace). These large-amplitude excitatory events are usually followed by high-frequency small-amplitude sEPSCs. In mice with hNSC implants these events were markedly reduced in frequency (second trace). (F) In cells with “epileptiform” activity (6-8 min after BIC), there was a rightward shift in the cumulative inter-event interval probability distributions for the hNSC-implanted R6/2 group compared with vehicle, corresponding to a significant decrease in high-frequency spontaneous events (p<0.001, two-way repeated-measures ANOVA followed by Bonferroni post hoc analysis; *p<0.05).

FIGS. 4A-4B: Nerve Terminals from the Host Make Synaptic Contact with the Implanted hNSCs. (A) Unlabeled nerve terminal (U-NT), containing synaptic vesicles, making a synaptic-like contact (arrow) with an underlying labeled (SC121) hNSC dendrite (L-DEND). The connection may be symmetrical. (B) Unlabeled nerve terminal (U-NT), containing synaptic vesicles, making an asymmetrical synaptic contact (arrow) with an underlying labeled (SC121) hNSC dendrite (L-DEND). This asymmetrical contact suggests an excitatory synaptic contact.

FIGS. 5A-5G: ESI-017 hNSCs Implanted in Q140 Mice Improve Behavior and Exhibit Evidence of Differentiation into Immature Neurons and Astrocytes. (A) Transient improvement in motor coordination (pole task) 3 months after cell injection. WT Veh (n=20), Q140 Veh (n=18), Q140 hNSC (n=18). One-way ANOVA with Bonferroni post hoc test: *p<0.05, **p<0.01. (B-D) Persistent improvement of running wheel deficits 5.5 months post treatment (n=5 per group). (B) Graph showing mean running wheel rotations/3 min/night over 2 weeks, in 7.5-month-old male WT or Q140 mice 5.5 months post treatment. Comparison by two way ANOVA: group effect F=52.93, p<0.0001; night in running wheel effect F=17, p<0.0001. Bonferroni post hoc test: *p<0.01, **p<0.001, and ***p<0.0001 compared with Q140 Veh. (C) Total average running wheel turns at night over 2 weeks. Two-way ANOVA with Bonferroni post hoc test: *p<0.01, **p<0.001. (D) Slope of motor learning not significant between the three groups. (E and F) Novel object recognition. hNSCs prevented the deficit in Q140 mice 5 months post treatment but not at 3 months in the discrimination index of sniffing time (E) or number of bouts (F). WT Veh n=18, Q140 Veh n=18, and Q140 hNSC n=19. One-way ANOVA with Bonferroni post hoc test: *p<0.05, **p<0.01. (G) Survival and differentiation of hNSCs in Q140 mice by staining with the human specific antibody (HNA; a and d) co-expressing with astrocytes (GFAP; b and c) or neuron-restricted progenitors (DCX; e and f). Scale bar, 20 mm. All graphs show mean±SEM.

FIGS. 6A-6D: ESI-017 hNSCs Implanted in HD Mice Increase Expression of BDNF. (A) ESI-017 hNSCs (Ku80) show co-localization with BDNF; astrocytes are shown as GFAP positive. (B) Veh-treated mice show no BDNF or hNSCs but have GFAP. (C) BDNF levels by ELISA in striatum of Q140 or WT mice 6 months post implant. (D) hNSC treatment in Q140 mice decreased microglial activation. Data are presented as the mean+95% confidence interval (n=5 per group). Bars represent percentage of cells of each diameter and the gray portion represents the confidence interval. Significant striatal microglial activation observed in Q140 Veh compared with WT Veh. Q140 hNSC mice showed significant reduction of microglial activation in striatum compared with Q140 Veh mice. *p<0.05 and **p<0.01 by one-way ANOVA with Bonferroni post hoc test. Graphs show means±SEM.

FIGS. 7A-7F: ESI-017 hNSCs Implanted in R6/2 Mice Cause Decreases in Diffuse Aggregates and Inclusions and Reduce Huntingtin Aggregates in Q140 Mice. (A and B) ESI-017 hNSCs cause decreases in diffuse aggregates and inclusions (arrows in A) in R6/2 mice. (A) Image of Ku80 with nickel, HTT marker EM48, and cresyl violet for non-hNSC nuclear staining. Stereological assessment performed using StereoInvestigator. Contour tracing under 53 objective (dashed lines, example in left panel) and counting at 1003. Every third section was counted (40-mm coronal sections) for 6 sections throughout the striatum where Ku80 could be seen between bregma 0.5 mm and bregma 0.34 mm. (B) Graph depicting percentage of cells with aggregates or inclusions (n=4/group) **p<0.01 by one-way ANOVA with Bonferroni post hoc test. (C and D) ESI-017 hNSCs reduce Huntingtin aggregates in Q140 mice. (C) Images of HTT marker EM48 (arrows indicate inclusions). (D) HTTstained nuclei and aggregates were analyzed with StereoInvestigator for quantification of aggregate type/section. Data are shown as mean±SEM (n=5/group). *p<0.05 by one-way ANOVA with Bonferroni post hoc test. (E and F) hNSC transplantation modulates insoluble protein accumulation in R6/2 mice. Western blot of striatal lysates separated into detergent-soluble and detergent-insoluble fractions. (E) R6/2 enriched in insoluble accumulated mHTT compared with NT. hNSC transplantation in R6/2 results in a significant reduction of insoluble HMW accumulated HTT compared with veh-treated animals. R6/2 striatum is also enriched in insoluble ubiquitin-conjugated proteins compared with NT. hNSC transplantation in R6/2 mice results in a significant reduction of ubiquitin-modified insoluble conjugated proteins compared with veh treatment with no significant effect in NT compared with veh controls. (F) Quantitation of the relative protein expression for mHTT and ubiquitin. Values represent means±SEM. Statistical significance for relative insoluble accumulated mHTT and ubiquitin-conjugated protein expression in R6/2 was determined with a one-way ANOVA followed by Bonferroni post hoc test (n=3/treatment). *p<0.05, **p<0.01, ***p<0.001. Graphs show means±SEM.

FIGS. 8A-8D: Characterization of ESI-017 hNSCs by Single Color Flowcytometry. (A) ESI-017 hNSCs stain positive for CD24, SOX1, SOX2, Nestin and Pax6 NSC markers. ESI-017 hNSCs stain negative for the pluripotent marker SSEA4. Karyotyping on ESI-017 hNSCs was performed and metaphases were visualized by Giemsa staining of condensed chromosomes. The final Karyotype was shown to have a high mitotic index with a 46 XX normal profile. (B) Flow Diagram of the NSC manufacturing process: hNSCs are generated by embryoid body (EB) formation, followed by plating of the generated EBs into poly-ornithin-laminin (Poly-O) coated plates with subsequent neural rosette formation. Rosettes are manually dissected and transferred into fresh Poly-O plates, where they are allowed to attach. Expanded neural rosettes are then enzymatically dissected, followed by plating into fresh Poly-O plates. There the cells are allowed to grow to confluence and are passaged enzymatically into larger number of Poly-O plates. Final harvest and cryopreservation of generated hNSCs is performed after expansion to sufficient numbers. (C) Cultured ESI-017 hNSC Immunocytochemistry shows positive NSC staining for neuralectodermal stem cell marker Nestin and DAPI nuclear staining. Scale bar equals 30 μm. (D) is a picture of a rosette.

FIG. 9: Clasping behavior: R6/2 mice treated with ESI-017 hNSCs (n=15) show delayed clasping behavior post implant. Non-transgenic (NT) mice do not demonstrate this phenotype. Mice were tested daily for the phenotype and graphs depict percentage of each group clasping over the course of the study. Significance in the clasping assay was determined by Fisher's exact probability test.

FIGS. 10A-10E: Low magnification Immunohistochemistry of ESI-017. hNSC implanted R6/2 mice: hNSCs (human marker SC121) implanted in R6/2 mice co-localize with marker for neuron restricted progenitors (doublecortin DCX). To screen for hNSC, IHC is performed on sections #34, 37, 40, 43, 46, and 49 (equivalent to Bregma 0.38 mm, 0.26 mm, 0.14 mm, 0.02 mm, −0.10 mm, and −0.22 mm, respectively). S2 is a re-use of the image shown in FIG. 1D for a comparison to other coronal sections. ESI-017 hNSC implant in R6/2 mice Immunohistochemistry: (A) hNSCs (human marker Ku80) implanted in R6/2 mice do not co-localize with an oligodendrocyte marker (Olig2) mouse cell nuclei shown with DAPI. High magnification (63×) showing differentiation: (B) hNSCs (human nuclear marker Ku80 and cytosolic marker SC121 blue) shows colocalization (lt. blue) with neuron restricted progenitors (BIII-tubulin). (C) hNSCs (human nuclear marker Ku80 and cytosolic marker SC121) shows co-localization with neuron restricted progenitors (MAP-2). (D) hNSCs (human nuclear marker Ku80) do not co-localize with huntingtin marker (EM48). (E) S1-6 shows coronal sections collected and immuno-stained starting at bregma 1.70 mm, 40 um per section.

FIGS. 11A-11B: ESI-017 hNSCs implanted into the striatum did not improve deficits in Open field or Climbing cage tests in Q140 mice. Mice were tested in the open field (A) for 15 minutes and climbing cage for 5 minutes (B) at 0.5 months pre-implant, or 3 and 5 months post implant. Data are represented as the mean±SEM; Wt Veh (n=18), Q140 Veh (n=18), and Q140 hNSC (n=17). Two-way ANOVA with Bonferroni post-test *p<0.05, ** p<0.01, *** p<0.001 compared to same time point of Vehicle-treated Wt mice.

FIGS. 12A-12C: ESI-017 hNSC BDNF expression in vitro. ESI-017 hNSCs were cultured in neural stem cell media. (A) or differentiated (B) then stained for BDNF human nuclear marker Ku80 and doublecortin DCX. (C) qPCR comparing RNA levels from cultured ESI-017 hNSCs show BDNF expression increased with differentiation. For comparison the stem cell marker nestin decreased with differentiation and DCX increased.

FIGS. 13A-13E: (A&B) Synaptophysin levels are increased in the striatum of Q140 mice with ESI-017 hNSCs. (A) Images were taken with a microarray scanner and quantified for fluorescence intensity. White scale bar equals 10 μm. (B) Data are shown as mean±SEM and statistical test used was One-way ANOVA with Bonferroni post-test *p<0.05, n=5 mice per group. hNSC treatment in R6/2 mice does not alter microglial activation. Data are represented as the mean+95% confidence interval (n=5 per group). Bars represent percent cells of each diameter and the colored portion represents the confidence interval. (C) Significant striatal microglial activation observed in R6/2 mice treated with vehicle (R6/2 Veh) compared to Non-transgenic control (NT Veh). (D) Comparison of NT+vehicle to NT+hNSCs. (E) R6/2 mice treated with hNSCs (R6/2 NSC) showed no significant reduction of microglial activation in striatum compared to R6/2 Veh mice.

FIG. 14: Real-time PCR of human HTT transgene expression in R6/2 mice. RPLPO (Large Ribosomal Protein) endogenous control was used to normalize gene expression differences in cDNA samples. No significance was observed as determined by one-way ANOVA with Bonferroni post-testing.

FIGS. 15A-15F: R6/1 mice were given bilateral intrastriatal injections of AAV expressing sApiCCT1 or mCherry control at 5 weeks of age. In two separate experiments, mice were injected with 12×10⁹ genome copies of AAV2/1 and harvested at 17 weeks of age. (A) Schematic. (B,C) Quantitation of agarose gel electrophoresis followed by western blot shows a significant reduction in oligomeric mHTT in animals. (D) Immunohistochemistry shows expression of sApiCCT1 (anti-HA). (E) sApiCCT1 injected mice show an approximate 40% reduction in visible mHTT inclusions by stereology (anti-EM48) (F) Mice injected with sApiCCT1-AAV2/1 show improvements on rotarod motor task *p<0.05, **p>0.01.

FIG. 16A-16D: ESI-017 hNSCs produce ApiCCT. (A) ESI-017 hNSCs transduced with sApiCCT lentivirus at MOI of 0, 5, 10 or 15 were cultured for 48 hours post transduction, lysed and Western blot performed using HA antibody then stripped and re-probed with alpha-Tubulin antibody for loading control. (B) ApiCCT secreted from hNSCs enters PC12 Htt14A2.6 cells. Conditioned media from ESI-017 hNSCs transduced with sApiCCT lentivirus was applied to 14A2.6 cells induced by ponasterone in EtOH to express HTT-GFP or controls treated with EtOH alone. ApiCCT1 is detected in cell lysates, supporting feasibility of engineering hNSCs to express a secreted form of ApiCCT1 that can be taken up by neighboring cells following transplantation. Western blot is shown using HA antibody. With higher MOI, higher amounts of ApiCCT1 is detected in treated PC12 cell lysates. (C) ApiCCT1 secreted from hNSCs does not alter monomeric HTT in PC12 Htt14A2.6 cells. Conditioned media from ESI-017 hNSCs transduced with sApiCCT lentivirus was applied to ponasterone-induced 14A2.6 cells or controls treated with EtOH alone. Treatment with secreted ApiCCT1 did not result in changes in monomeric mHTT-GFP transgene. Western blot shown using GFP antibody then stripped and re-probed for alpha-tubulin as loading control. (D) ApiCCT1 secreted from hNSCs alters oligomeric HTT species in PC12 Htt14A2.6 cells. Conditioned media from ESI-017 hNSCs transduced with sApiCCT1 lentivirus and applied to ponasterone-induced14A2.6 cells or controls treated with EtOH alone caused reduction of oligomeric HTT at the highest MOI (red box). Western blot of representative sample shown using GFP antibody.

FIGS. 17A and 17B: IHC Shows that ESI-017 hNSCs transduced with virus for ApiCCT and Implanted in the striatum of R6/2 Mice Express ApiCCT. (A) hNSCs (Human Nuclear Antigen [HNA]) implanted in R6/2 mice differentiate into neuron-restricted progenitors (doublecortin [DCX]) and express HA tagged ApiCCT (HA). (B) High magnification (95×) taken from area in white box indicated in A showing differentiation and ApiCCT expression: hNSCs (HNA) implanted in R6/2 mice differentiate into neuron-restricted progenitors (DCX) and express HA tagged ApiCCT (HA).

### DETAILED DESCRIPTION

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

Throughout and within this application technical and patent literature are referenced by a citation. For certain of these references, the identifying citation is found at the end of this application immediately preceding the claims. All publications are incorporated by reference into the present disclosure to more fully describe the state of the art to which this disclosure pertains.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The term ‘isolating” intends the process of separating a composition or component from others in close proximity or contingent therewith. Cells can be isolated manually (e.g., by hand using a pipette or other tool), enzymatically by the use of chemical agents or digitally by the use of digital techniques based on cell or rosette morphology. See, e.g., cellavision.com/en/introducing-digital-cell-morphology-by-cellavision, accessed on May 22, 2018.

“Differentiation medium” intends cell culture medium that contains factors, such as certain growth factors, that promote the differentiation of an immature cell to a more mature phenotype, e.g., from an embryonic stem cell to a neural cell.

As used herein, the term “confluent population” intends a population of cells that are in contiguous contact with the adjacent cells.

An “ultra-low attachment surface” intends cell or tissue culture surfaces that in some aspects, contain a covalently bound hydrogel layer that is hydrophilic and neutrally charged. Since proteins and other biomolecules passively adsorb to polystyrene surfaces through either hydrophobic or ionic interactions, this hydrogel surface naturally inhibits nonspecific immobilization via these forces, thus inhibiting subsequent cell attachment. These surfaces are commercially available from a variety of vendors, e.g. Millipore-Sigma, Fisher-Scientific, and S-bio. Methods are known in the art for manufacturing cell culture plates and surfaces.

A “transgene” intends a polynucleotide that has been added to a cell, a tissue or organism. An example of a transgene is ApiCCT1.

“ApiCCT1” refers to the apical domain of CCT1 and/or a polynucleotide encoding said apical domain of CCT1 (Sontag, E. Proc Natl Acad Sci USA. 2013 Feb. 19; 110(8):3077-82, incorporated herein by reference). CCT1 is a molecular chaperone that is a member of the chaperonin containing TCP1 complex (CCT), also known as the TCP1 ring complex (TRiC). This complex consists of two identical stacked rings, each containing eight different proteins. Unfolded polypeptides enter the central cavity of the complex and are folded in an ATP-dependent manner. The complex folds various proteins, including actin and tubulin. In some embodiments, the ApiCCT1 is 20 kDa in size. In humans, the TCP1-ring complex is encoded by the TCP1 gene (Entrez gene 6950). Non-limiting examples of the sequence of TCP1 mRNA and protein are provided herein as SEQ ID NOs.: 1-4. The apical domain is involved in substrate binding. (Pappenberger, G. et al. J Mol Biol. 2002 May 17; 318(5):1367-79, incorporated herein by reference). A non-limiting example of the sequence of ApiCCT1 is provided below (SEQ ID NO: 7):

MVPGYALNCTVASQAMPKRIAGGNVKIACLDLNLQKARMAMGVQINIDDP
EQLEQIRKREAGIVLERVKKIIDAGAQWLTIKGIDDLCLKEFVEAK1MGV
RRCKKEDLRRIARATGATLVSSMSNLEGEETFESSYLGLCDEWQAKFSDD
ECILIKGTSKAAAAALE.

“sApiCCT1” refers to a secreted version of ApiCCT1. Non-limiting examples of a nucleic acid sequence and an amino acid sequence of sApiCCT are provided below. The underlined sequences correspond to an HA tag. In some embodiments, sApiCCT1 does not comprise a tag.

sApiCCT1 mRNA
(SEQ ID NO: 8)
ATGTACAGGATGCAACTCCTGTCTTGCATTGCACTAAGTCTTGCACTTGT
CACGAATTCTATCAGTGGCTATGCACTCAACTGTGTGGTGGGATCCCAGG
GCATGCCCAAGAGAATCGTAAATGCAAAAATTGCTTGCCTTGACTTCAGC
CTGCAAAAAACAAAAATGAAGCTTGGTGTACAGGTGGTCATTACAGACCC
TGAAAAACTGGACCAAATTAGACAGAGAGAATCAGATATCACCAAGGAGA
GAATTCAGAAGATCCTGGCAACTGGTGCCAATGTTATTCTAACCACTGGT
GGAATTGATGATATGTGTCTGAAGTATTTTGTGGAGGCTGGTGCTATGGC
AGTTAGAAGAGTTTTAAAAAGGGACCTTAAACGCATTGCCAAAGCTTCTG
GAGCAACTATTCTGTCAACCCTGGCCAATTTGGAAGGTGAAGAAACTTTT
GAAGCTGCAATGTTGGGACAGGCAGAAGAAGTGGTACAGGAGAGAATTTG
TGATGATGAGCTGATCTTAATCAAAAATACTAAGGCTGCTGCGGCTGCGG
GTGGACACTACCCTTACGACGTGCCTGACTACGCCTGA
sApiCCT1 peptide
(SEQ ID NO: 9)
MYRMQLLSCIALSLALVTNSISGYALNCVVGSQGMPKRIVNAKIACLDFS
LQKTKMKLGVQVVITDPEKLDQIRQRESDITKERIQKILATGANVILTTG
GIDDMCLKYFVEAGAMAVRRVLKRDLKRIAKASGATILSTLANLEGEETF
EAAMLGQAEEVVQERICDDELILIKNTKAAAAAGGHYPYDVPDYA

As used herein, “BDNF” intends brain derived neurotrophic factor (BDNF) and equivalents thereof and/or a polynucleotide encoding BDNF or equivalents thereof. BDNF acts on neurons of the central nervous system and the peripheral nervous system, helping to support the survival of existing neurons, and encourage the growth and differentiation of new neurons and synapses. BDNF is also active in the hippocampus, cortex, and basal forebrain—areas vital to learning, memory, and higher thinking. It is also expressed in the retina, motor neurons, the kidneys, saliva, and the prostate. The BDNF protein is encoded by the BDNF gene (Entrez gene: 627; mRNA: NM_001143805, NM_001143806, NM_001143807, NM_001143808, NM_001143809, NM_001143810, NM_001143811, NM_001143812, NM_001143813, NM_001143814, NM_001143815, NM_001143816, NM_001709, NM_170731, NM_170732, NM_170733, NM_170734, NM_170735). Non-limiting examples of BDNF mRNA and protein sequences are provided herein as SEQ ID NOs: 5-6.

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of nonhomologous end-joining (NHEJ) or homologous recombination to perform the edits. Gene regulation refers to increasing or decreasing the production of specific gene products such as protein or RNA.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83). As used herein, a biological equivalent of a gRNA includes but is not limited to polynucleotides or targeting molecules that can guide a Cas9 or equivalent thereof to a specific nucleotide sequence such as a specific region of a cell's genome.

Expression of CRISPR in cells can be achieved using conventional CRISPR/Cas systems and guide RNAs specific to the target genes in the cells. Suitable expression systems, e.g. lentiviral or adenoviral expression systems are known in the art. It is further appreciated that a CRISPR editing construct may be useful in both knocking out an endogenous gene or knocking in a gene. Accordingly, it is appreciated that a CRISPR system can be designed for to accomplish one or both of these purposes.

As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

An equivalent or biological equivalent nucleic acid, polynucleotide or oligonucleotide or peptide is one having at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, oligonucleotide or peptide.

The term “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to and hybridize specifically to sequences in the target region or its flanking regions. Nucleic acid sequences generated by amplification may be sequenced directly. Alternatively, the amplified sequence(s) may be cloned prior to sequence analysis. A method for the direct cloning and sequence analysis of enzymatically amplified genomic segments is known in the art.

A “gene” refers to a polynucleotide containing at least one open reading frame (ORF) that is capable of encoding a particular polypeptide or protein after being transcribed and translated.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell. In one aspect, this invention provides promoters operatively linked to the downstream sequences, e.g., suicide gene, a polynucleotide encoding ApiCCT1, a fragment thereof such as sApiCCT1, or an equivalent of each thereof.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “probe” when used in the context of polynucleotide manipulation refers to an oligonucleotide that is provided as a reagent to detect a target potentially present in a sample of interest by hybridizing with the target. Usually, a probe will comprise a detectable label or a means by which a label can be attached, either before or subsequent to the hybridization reaction. Alternatively, a “probe” can be a biological compound such as a polypeptide, antibody, or fragments thereof that is capable of binding to the target potentially present in a sample of interest.

“Detectable labels” or “markers” include, but are not limited to radioisotopes, fluorochromes, chemiluminescent compounds, dyes, and proteins, including enzymes. Detectable labels can also be attached to a polynucleotide, polypeptide, antibody or composition described herein.

A “primer” is a short polynucleotide, generally with a free 3′ —OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). All processes of producing replicate copies of a polynucleotide, such as PCR or gene cloning, are collectively referred to herein as “replication.” A primer can also be used as a probe in hybridization reactions, such as Southern or Northern blot analyses. Sambrook and Russell (2001), infra.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Additional examples of stringent hybridization conditions include: low stringency of incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

The term “propagate” or “expand” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, bacculoviruses, modified bacculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing non-dividing cells as compared to other retroviral vectors. See, Trono D. (2002) Lentiviral vectors, New York: Spring-Verlag Berlin Heidelberg.

Lentiviral vectors of this invention are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the invention may be based on a genetically or otherwise (e.g. by specific choice of packaging cell system) altered version of a particular retrovirus.

That the vector particle according to the invention is “based on” a particular retrovirus means that the vector is derived from that particular retrovirus. The genome of the vector particle comprises components from that retrovirus as a backbone. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

The term “promoter” refers to a region of DNA that initiates transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible. A constitutive promoter refers to one that is always active and/or constantly directs transcription of a gene above a basal level of transcription. Non-limiting examples of such include the phosphoglycerate kinase 1 (PGK) promoter; SSFV, CMV, MNDU3, SV40, Ef1a, UBC and CAGG. An inducible promoter is one which is capable of being induced by a molecule or a factor added to the cell or expressed in the cell. An inducible promoter may still produce a basal level of transcription in the absence of induction, but induction typically leads to significantly more production of the protein. Promoters can also be tissue specific. A tissue specific promoter allows for the production of a protein in a certain population of cells that have the appropriate transcriptional factors to activate the promoter.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. A clone is a line of cells that is genetically identical to the originating cell; in this case, a stem cell.

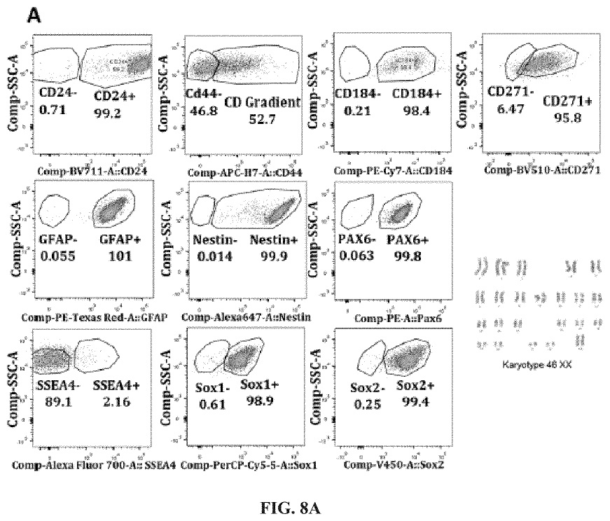

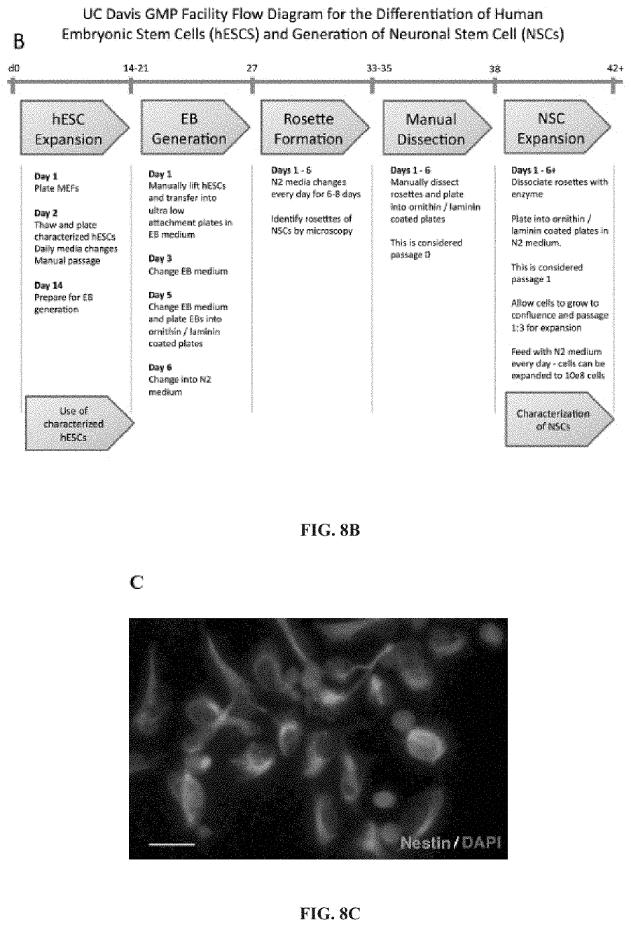

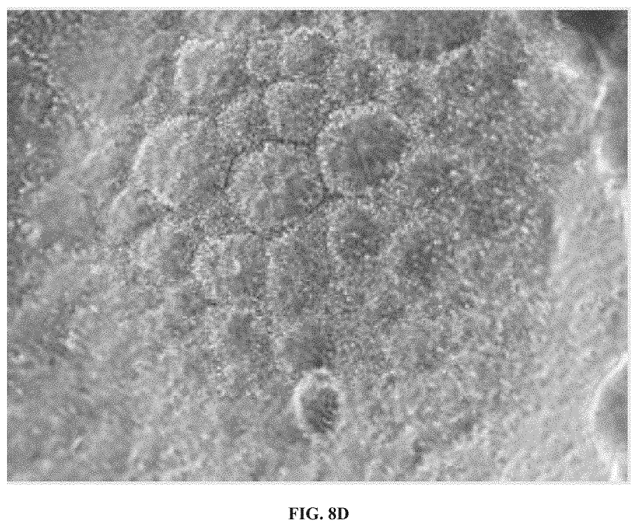

A “stem cell rosette” intends a cluster of stem cells that, under magnification, appears as a cluster of petals. See, for example, FIG. 8D.

A population of cells intends a collection of more than one cell that is identical (clonal) or non-identical in phenotype and/or genotype. A substantially homogenous population of cells is a population having at least 70%, or alternatively at least 75%, or alternatively at least 80%, or alternatively at least 85%, or alternatively at least 90%, or alternatively at least 95%, or alternatively at least 98% identical phenotype, as measured by pre-selected markers.

As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

A neural stem cell is a cell that can be isolated from the adult central nervous systems of mammals, including humans. They have been shown to generate neurons, migrate and send out axonal and dendritic projections and integrate into pre-existing neuronal circuits and contribute to normal brain function. Reviews of research in this area are found in Miller (2006) The Promise of Stem Cells for Neural Repair, Brain Res. Vol. 1091(1):258-264; Pluchino et al. (2005) Neural Stem Cells and Their Use as Therapeutic Tool in Neurological Disorders, Brain Res. Brain Res. Rev., Vol. 48(2):211-219; and Goh, et al. (2003) Adult Neural Stem Cells and Repair of the Adult Central Nervous System, J. Hematother. Stem Cell Res., Vol. 12(6):671-679.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

As used herein, the term “differentiates or differentiated” defines a cell that takes on a more committed (“differentiated”) position within the lineage of a cell. “Dedifferentiated” defines a cell that reverts to a less committed position within the lineage of a cell. Induced pluripotent stem cells are examples of dedifferentiated cells.

As used herein, the “lineage” of a cell defines the heredity of the cell, i.e. its predecessors and progeny. The lineage of a cell places the cell within a hereditary scheme of development and differentiation.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multi-lineage stem cell is a myogenic cell and an adipogenic cell (both are of mesodermal origin yet give rise to different tissues). Another example is a neurogenic cell (of ectodermal origin) and adipogenic cell (of mesodermal origin).

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell includes, without limitation, a progenitor nerve cell.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100(Suppl. 1)11911-6.

As used herein, a “pluripotent cell” defines a less differentiated cell that can give rise to at least two distinct (genotypically and/or phenotypically) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

“Embryoid bodies or EBs” are three-dimensional (3D) aggregates of embryonic stem cells formed during culture that facilitate subsequent differentiation. When grown in suspension culture, EBs cells form small aggregates of cells surrounded by an outer layer of visceral endoderm. Upon growth and differentiation, EBs develop into cystic embryoid bodies with fluid-filled cavities and an inner layer of ectoderm-like cells.

A “composition” is intended to mean a combination of active polypeptide, polynucleotide or antibody and another compound or composition, inert (e.g. a detectable label) or active (e.g. a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like which is susceptible to neurodegenerative disease. In one embodiment, the mammals include horses, dogs, and cats. In another embodiment of the present invention, the human is an adolescent or infant under the age of eighteen years of age.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “suffering” as it related to the term “treatment” refers to a patient or individual who has been diagnosed with or is predisposed to infection or a disease incident to infection. A patient may also be referred to being “at risk of suffering” from a disease because of active or latent infection. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to inhibit RNA virus replication ex vivo, in vitro or in vivo.

The term administration shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository), intracranial, or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

Huntington's disease (HD) is an inherited disease that causes the progressive breakdown (degeneration) of nerve cells in the brain. Huntington's disease has a broad impact on a person's functional abilities, including loss of motor and cognitive function as well as psychiatric disorders. To treat or ameliorate the symptoms of HD intends to improve the patient's, psychiatric, cognitive or motor function or reduce the adverse effect of this inherited disorder. The symptoms and course of the disease are known to the skilled artisan, see mayoclinic.org/diseases-conditions/huntingtons-disease/symptoms-causes/syc-20356117, accessed on May 21, 2018.

A central nervous system (CNS) disease or disorder intends a group of neurological disorders that affect the structure of function of the brain or spinal cord, and that may result in degeneration of one or more parts of the brain or spinal cord. Non-limiting examples include HD, Alzheimer's disease, Parkinson's disease, traumatic brain injury, stroke, autoimmune disorders such as multiple sclerosis, primary or secondary progressive multiple sclerosis, relapsing remitting multiple sclerosis, brain inflammation, Bell's palsy, cervical spondylosis, carpal tunnel syndrome, brain or spinal cord tumors, peripheral neuropathy, Guillain-Barre syndrome, spinal muscular atrophy, Freidrich's ataxia, amyotrophic lateral sclerosis, and Huntington chorea. To treat or ameliorate the symptoms of a CNS injury intends to improve the patient's nerve function reduce the adverse effect of inherited or acquired disease, injury or a disorder. The symptoms and course of the disease are known to the skilled artisan, see, hopkinsmedicine.org/healthlibrary/conditions/nervous_system_disorders/overview_of_nervo us_system_disorders 85,P00799, accessed on May 21, 2018.

A “neurodegenerative disease or disorder” is a disease or phenotype characterized by degeneration of the nervous system, especially the neurons in the CNS.

“To enhance synaptic connections” intends to promote connections between neurons or neuronal receptors.

A synapse is a junction between two nerve cells, consisting of a minute gap across which impulses pass by diffusion of a neurotransmitter.

Modes for Carrying Out the Disclosure

Provided herein is a method to prepare a human neuronal stem cell (hNSC) from a human embryonic stem cell (hESC), the method comprising, or alternatively consisting essential of, or yet further consisting of, the steps of:
a) isolating at least one stem cell rosette from a population of embryoid bodies (EB) cultured in differentiation medium;
b) culturing at least one individual cell isolated from the rosette of step a) for an amount of time and under until conditions that provide for the generation of at least one rosette;
c) isolating an individual cell from the rosette of step b) into individual cells; and
d) culturing the at least one individual cell isolated from step c) for an amount of time and under until conditions that provide for the generation of confluent population of hNSCs.

In one aspect, the isolation of the at least one individual cell from the rosette is performed manually. In another aspect, the isolation of the at least one individual cell from the rosette is performed enzymatically. In a further aspect, the isolation of the at least one individual cell from the rosette of step a) is performed by one or more of: manually, enzymatically, and/or digitally. In a yet further aspect, the isolation of the at least one individual cell of step c) is performed enzymatically. Methods and techniques to digitally identify a three- or two-dimensional image are known in the art, see for example, U.S. Pat. Nos. 7,689,043; 6,907,140; and 5,020,112.

In one embodiment, the one or more of steps a) through c) is performed 2 or more times that can be performed using one or both of manually, or mechanically in a high throughput manner. In a further aspect, the isolation of the rosette is performed digitally. Methods and techniques to digitally identify a three- or two-dimensional image are known in the art, see for example, U.S. Pat. Nos. 7,689,043; 6,907,140; and 5,020,112.

In one aspect, the embryoid bodies are generating from the cell line ESI-017, available from BioTime (see, esibio.com/esi-017-human-embryonic-stem-cell-line-46-xx/, last accessed on Jun. 6, 2018).

In one aspect of the disclosure, the method further comprises culturing the embryoid body (EB) on an ultra-low attachment surface in EB medium. In another aspect, the method further comprises substituting N2 medium for the EB medium after the EBs have been cultured for an effective amount of time further to step a) on an ornithine/laminin coated surface. Alternatively, the method further comprises substituting N2 medium for the EB medium after the EB have been cultured in the EB medium for an amount of time effective to produce at least one EB of step a).

The methods can be further modified by having at least one individual cell isolated in step c) cultured for an effective amount of time on an ornithin/laminin coated plate in N2 medium to generate a confluent cell population of hNSCs. As is known to those of skill in the art, a confluent cell population is one wherein a substantial number of the cells are in contact with others in the population. This method can be further modified by culturing the confluent population of hNSCs with an effective amount of N2 medium.

Also provided herein is a cell or a population of cells prepared by the methods as described herein. The neuronal cells and the differentiated cells of produce BDNF or overexpress BDNF.

The cells of the population can be expanded and/or genetically modified by, for example, by insertion of a transgene or by CRISPR. In one aspect, the transgene is ApiCCT1, a fragment thereof such as sApiCCT, or an equivalent of each thereof. The cells and/or transgene can optionally be detectably labeled. The transgene can be inserted using well known and conventional recombinant techniques by inserting the transgene in a vector, the transgene being under the control of regulatory elements, such as a promoter and optionally, an enhancer element. The cells and/or vectors containing the transgene can be detectable labeled. As detailed below, the transgene sApiCCT is inserted into specific cell populations of the hNSCs to offer further protection to the hNSCs or to tissue when implanted as a therapeutic. The sApiCCT transgene can also be inserted into hESCs or other stem cell derivatives including but not limited to other embryonic cell lines, fetal derived cell lines, mesenchymal derived cell lines, neuronal derived cell lines, as well as differentiated cell types.

A population of these cells are further provided, as well as non-human animals comprising the cells. The populations can be substantially homogenous, substantially heterogeneous or clonal. The populations can be detectably labeled. The populations can be combined with a carrier such as a pharmaceutically acceptable carrier.

Compositions comprising the isolated cells are further provided, with for example a carrier. In a further aspect, the composition further comprises a preservative and/or cryoprotectant. Non-limiting examples of cryoprotectants include DMSO, glycerol, that are commercially available, see e.g., streck.com/collection/streck-cell-preservative/, last accessed on May 22, 2018.

The cells are useful in therapeutic methods. In one aspect, methods are provided to deliver a transgene to a subject, or to genetically edit a cell in a subject in need thereof, by administering an effective amount of one or more of a cell, a population or a composition as described herein. In another aspect, methods of treating a neurodegenerative disorder or enhancing synaptic connections in a subject in need thereof are provided by administering to the subject an effective amount of one or more of a cell, a population or a composition. In another aspect, methods of treating a neurodegenerative disorder or enhancing synaptic connections or treating a CNS injury in a subject in need thereof are provided, comprising administering to the subject an effective amount of one or more of a cell, a population or a composition to the subject. Any appropriate method of administration can be used, non-limiting examples of such are provided herein.

Non-limiting examples of neurodegenerative disorders are selected from the group of Huntington's disease, stroke, CNS injury, chronic spinal cord injury, spinal cord injury, aneurism, surgery, arteriovenous malformation (AVM), radiation, spinal muscular atrophy, Freidrich's ataxia, amyotrophic lateral sclerosis (ALS), muscular sclerosis, primary or secondary progressive multiple sclerosis, relapsing remitting multiple sclerosis, vascular dementia, epileptic seizures, cerebral vasospasm, Alzheimer's disease, acute or traumatic brain injury, brain inflammation, and hypoxia of the brain as a result of, for example, cardiopulmonary arrest or near drowning or any other CNS injury resulting in acute physical damage to CNS tissue and combinations thereof.

In certain embodiments, the CNS injury is one that has been caused by a stroke. By “stroke” is meant, any condition that results in physical damage to the central nervous system due to disturbance in the blood supply or oxygen to the brain. This can be due to ischemia (lack of blood supply or oxygen) caused by thrombosis or embolism or due to a hemorrhage.

### Kits

Kits also are provided. In one aspect, the kit comprises an hESC and instructions to perform the methods as described herein. In a further aspect, the kit comprises a neuronal cell prepared using the methods as described herein and instructions for use. The kits can further comprise compositions and reagents to carry out the instructions provided with the kits.

The agents described herein may, in some embodiments, be assembled into pharmaceutical or diagnostic or research kits to facilitate their use in therapeutic, diagnostic or research applications. In one aspect, the kit comprises an hESC and instructions to perform the methods as described herein. In a further aspect, the kit comprises a neuronal cell prepared using the methods as described herein and instructions for use. The kits can further comprise compositions and reagents to carry out the instructions provided with the kits.

In some embodiments, a kit further comprises instructions for use. Specifically, such kits may include one or more agents described herein, along with instructions describing the intended application and the proper use of these agents. As an example, in one embodiment, the kit may include instructions for mixing one or more components of the kit and/or isolating and mixing a sample and applying to a subject. In certain embodiments, agents in a kit are in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In some embodiments, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In some embodiments, the preservation solution contains an amount of metalloprotease inhibitors.

As used herein, “instructions” can define a component of instruction and/or promotion, and typically involve written instructions on or associated with packaging of the claimed method or composition. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. In some embodiments, the written instructions is in a form prescribed by a governmental agency regulating the manufacture, use or sale of pharmaceuticals or biological products, which instructions can also reflect approval by the agency of manufacture, use or sale for animal administration.

In some embodiments, the kit contains any one or more of the components described herein in one or more containers. Thus, in some embodiments, the kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to a subject, such as a syringe, topical application devices, or IV needle tubing and bag.

The therapies as described herein can be combined with appropriate diagnostic techniques to identify and select patients for the therapy. For example, a genetic test to identify a mutation in a muscular dystrophy gene can be provided. Thus, patients harboring a mutation can be identified as suitable for therapy.

The following examples are intended to illustrate, and not limit this disclosure.

### Experimental Procedures

TABLE 1
Generation of ESI-017 hNSCs - Materials

| | | | | | Final |
|---|---|---|---|---|---|
| Description | Company | Cat. No | 250 ml | | Conc. |
| KO DMEM/F-12 | Life | 12660-012 | 195 | ml | |
| | Technologies | | | | |
| GlutaMAX ™ | Life | 35050-061 | 2.5 | ml | 1x |
| | Technologies | | | | |
| NEAA | Life | 11140-050 | 2.5 | ml | 1x |
| | Technologies | | | | |
| 2-Mercaptoethanol | Life | 21985-023 | 455 | ul | 0.2% |
| | Technologies | | | | |
| Knockout Serum | Life | 10828-028 | 50 | ml | 20% |
| Replacement (KSR) | Technologies | | | | |

TABLE 2
N2 Medium Recipe

| | | | | | Final |
|---|---|---|---|---|---|
| Description | Company | Cat. No | 50 ml | | Conc. |
| Cellgro DMEM/F12 | Corning | 15090-cv | 48.8 | mL | |
| GlutaMAX ™ | Life | 35050-061 | 0.5 | mL | 1x |
| | Technologies | | | | |
| N2 | Life | 17502-048 | 0.25 | mL | 0.5% |
| | Technologies | | | | |
| B27 | Life | 17504-044 | 0.5 | mL | 1% |
| | Technologies | | | | |
| bFGF (10 ug/ml) | Life | PHG0021 | 0.1 | mL | 20 ng/ml |
| | Technologies | | | | |

TABLE 3
Cryopreservation Medium Recipe

| | | | | | Final |
|---|---|---|---|---|---|
| Description | Company | Cat. No | Dilution | | Conc. |
| N2 media | Stock | NA | 9 | parts | 90% |
| DMSO | Sigma | D1435-1L | 1 | part | 10% |

TABLE 4
Additional Reagents

| | | | | Final |
|---|---|---|---|---|
| Description | Company | Cat. No | Dilution | Conc. |
| Murine Laminin | Sigma | L2020-1MG | 10 ul/mL | 10 ug/ml |
| | | | in PBS | |
| Poly-L-Ornithine | Sigma | P4957 | 1:3 in PBS | 25% |
| (poly-L) | | | | |
| 0.05% Trypsin | Life | 25300-054 | 1 ml/well | 100% |
| | Technologies | | | |
| Defined Trypsin | Life | R-007-100 | 1 ml/well | 100% |
| Inhibitor (DTI) | Technologies | | | |

TABLE 5
Solution Preparations
Preparation

| | |
|---|---|
| 1. | Dilute stock poly-L-ornithine 1:3 in PBS−/− |
| | (1 part poly-L to 2 parts PBS) |
| 2. | Dilute laminin 10 ul stock laminin per 1 ml PBS−/− |
| | (10 ug/ml) |
| 3. | bFGF − dilute 100 ug vial in 10 ml water = |
| | 10 ug/ml or 10 ng/ul |

Procedure for the Generation of NSCs from ESCs

### Passaging ESCs for Embryoid Body (EB) Formation.

On Day 1, Differentiated colonies were manually cleaned out from ESC cultures using a P1000 tip. The ESC medium was then changed from ESC cultures to 2 mL/well of EB medium. Using a P1000 tip, ESC colonies were scratched off with a back and forth motion, first horizontally across the wells, then vertically. Each well of scraped colonies was transferred with a 10 mL serological pipette into one well of an ultra-low attachment 6 well plate. The wells of the ESC plate were cleaned with a P1000 micropipetter using EB medium and wash was added to the respective wells of the ultra-low attachment 6 well plate to give a final volume of 3 mL/well. EB plates were moved to the incubator at 37° C. and 5% CO₂. The EB plats were incubated 37° C. and 5% CO₂ throughout Day 2. On Day 3, A half EB media change was performed. The floating colonies were gently swirled to the centers of their wells. Using a P1000 micropipettor, 1 ml of media was removed from each well and discarded. 1.5 mL of fresh EB medium was gently added back into to each well. Plate(s) were then returned to the incubator. On Day 4, there was no action. Stirring was continued while incubating at 37° C. and 5% CO₂. On Day 5, the EBs derived from ESCs were plated onto laminin coated plates. The appropriate number of wells in a 6 well plate were coated with poly-L-ornithine, diluted 1:3 in PBS, for one hour at room temperature. An N2 medium was prepared according to Table 2 (N2 media is good one week after preparation). After 1 hour, the poly-L-ornithine solution was removed and discarded. Wells were washed with PBS twice. Wells were coated with laminin and diluted 1:100 in PBS for one hour at room temperature. The suspension of EBs was transferred using a 10 mL serological pipette from each well into its own 15 mL conical tube. The EBs were allowed to settle out of suspension for 15 minutes at room temperature. For the wells of the coated 6 well plates, laminin was aspirated and discarded. 1 mL of N2 medium was added to each well. EB medium was aspirated from each 15 mL conical tube. EBs were gently resuspended in 2 mL of N2 medium using a 10 mL serological pipette. EBs were added to coated wells containing 1 mL of N2 medium for a total volume of 3 mL. The plates were gently rocked back and forth to distribute the EBs and placed into the incubator. On Days 6 through 14, rosette formation and isolation of the plated EBs (Ros1, round 1) were performed. Cells were inspected over the next 4-6 days to check for the formation of rosettes. Rosettes were picked at any time depending on quality of the formation. If there was not rosette formation yet, the N2 medium was changed every other day until rosettes appeared. For picking rosettes, a 12 well plate was coated with poly-L-ornithine, diluted 1:3 in PBS, for one hour at room temperature. After 1 hour, the poly-L-ornithine solution was removed and discarded. Wells were washed 3× with PBS. Wells were then coated with laminin, diluted 1:100 in PBS for one hour at room temperature. If needed, a bottle of N2 media was prepared according to Table 2. After 1 hour, laminin was removed and 1 mL N2 media was added to each well to keep moist and stored in the incubator for dissection of rosettes. Around day 10-12 rosettes can be seen that formed from EBs previously plated. Under a dissecting microscope, the rosettes were dissected out by tracing the rosette using an 18 gauge needle attached to a 1 mL syringe. The mixture was then transferred to the laminin coated plates in N2 media using a p200 micropipettor. After the transfer, the mixture was stored in the incubator overnight and labeled as Ros1. Media was changed every other day during storage in the incubator. On Days 15 through 18, the rosettes were dissociated into single cells. Two to three days after isolating Ros1's (round 1), the cleanest rosettes were dissociated into single cells. N2 media was aspirated from each desired well and 0.5 mL 0.05% trypsin in EDTA was added to each well. The mixture was incubated at 37° C. and 5% CO₂ for 90 seconds. After 90 seconds, 0.5 mL of DTI was added. Using a 1000 μL micropipettor, the rosette was dissociated in the well and the volume of each well was added to its own 15 mL conical tube. Each well, or “clone” was kept separate through all passages. The conical tube was spun down for 5 minutes at 1000 RPM. The supernatant was added and discarded. Each pellet was resuspended in 1 mL of fresh N2 medium. Each 1 mL suspension was plated onto its own well of a poly-L/laminin coated 12-well plate and the plates were labeled as (NSCs Passage 0). Plates were returned to the incubator and cultures monitored, media was changed every other day with fresh N2 medium. When cells reached about 85% confluence each “clone” was passed to its own well of a 6-well plate using the same procedure as above, but with 1 mL 0.05% trypsin and 1 mL DTI, and the media was changed every other day with 3 mL N2 media. Cells were maintained and passage continued at a ratio of 10⁶ cells/well, or cryopreservation of cells (discussed below) was undertaken.

### Cryopreservation of NSCs.

Cryopreservation media, or freezing media, was prepared according to Table 3, making sure that freezing media was chilled at 4° C. at all times before use. NSC plates were retrieved from the incubator and placed inside the biosafety cabinet. The old culture media was aspirated off and discarded into a waste container. 1 mL of 0.05% trypsin was added to each well and incubated at 37° C. for 90 seconds. After 90 seconds 1 mL DTI was added to each well to inactivate the trypsin. Using a 1000 μL pipette tip, the mixture was pipetted up and down to wash the cells off of the surface of each well and then the mixture transferred to a 15 mL conical tube. The 15 mL conical tubes were spun down for 3 minutes at 1000 RPM. Conical tubes were returned to the biosafety cabinet and the supernatant was aspirated off. Cells were resuspended in 5 mL of fresh N2 culture media and cell counts performed using 0.4% trypan blue and a hemocytometer. The cells were spun down at 1000 rpm for 3 minutes in a table top centrifuge. Appropriate volume of 4° C. freezing media was added to the cells so that cell concentration was at 3.0×10⁶ cells/mL. 1 mL of cell suspension in freezing media was added to each cryovial using a 10 mL serological pipette. One vial of freezing media with no cells was made up for the freezing probe. The vials were capped tightly and immediately transferred into the pre-chilled control rate freezer-freezing rack. The probe was inserted into the vial containing freeze media only and placed into the rack. Vials were transferred from the control rate freezer into pre-chilled −80° C. fully labeled cryoboxes and immediately transferred to LN2 storage.

### Mice

All procedures were in accordance with Guide for the Care and Use of Laboratory Animals of the NIH and approved animal research protocols by Institutional Animal Care and Use Committees at UCI and UCLA, AAALAC accredited institutions. R6/2 mice and their NT littermates (Transgene non-carrier C57B16/CBA) were obtained from breeding colonies maintained at UCI (line 6494, ˜120±5 CAG repeats) or UCLA (line 2810,˜150±5 CAG repeats). Homozygous Q140 mice or WT (C57B16) littermates were from breeding colonies at UCLA, where procedures were performed. All mice were housed on 12/12-hr light/dark schedule with ad libitum access to food and water. Mice were group housed as mixed treatment groups and only singly housed for the running wheel. CAG repeat length was confirmed for R6/2 mice (Laragen, Los Angeles, Calif.), and for Q140 mice frequency distributions are not significantly different (Hickey et al., 2012b). Assessment of differences in outcome were based upon previous experience and published results (Hickey et al., 2005; Hockly et al., 2003) for HD models, and applying power analysis (G Power [psycho.uni-duesseldorf.de/abteilungen/aap/gpower3/]) led Applicants to a minimal n=10 for behavior and n=4 for biochemical analysis.

hNSC Isolation

The use of hNSCs was approved by UCI's, UCLA's, and UC Davis' Human Stem Cell Research Oversight Committee (hSCRO) and cells were derived from Biotime ESI-017 hESCs. hESC colonies were transferred to EB medium with Noggin, transitioned to NP medium, and the rosettes dissected out, dissociated, and plated down with hNSC medium to generate hNSCs (FIG. 8B). Rosettes were manually dissected out and plated into growth factor-reduced Matrigel-coated plates in NSC medium then dissociated using Accutase and plated onto polyornithine/laminin-coated plates with NSC medium.

### Transplantation Surgery

Bilateral intrastriatal injections of hNSCs or veh were performed using a stereotactic apparatus and coordinates relative to bregma: anteroposterior, 0.00; mediolateral, ±2.00; dorsoventral, −3.25. Mice were anesthetized, placed in the stereotactic frame, and injected with either 100,000 hNSCs/side (2 μL/injection) or veh (2 μL Hank's balanced salt solution with 20 ng/mL human epidermal growth factor [STEMCELL Technologies, #78003] and human fibroblast growth factor [STEMCELL, #78006]) using a 5-4, Hamilton microsyringe (33-gauge) and injection rate 0.5 μL/min. Wounds were sealed and the mice recovered in cages with heating pads. Immunosuppressants were administered the day before surgeries to all mice and continued throughout.

### Behavioral Assessment

### R6/2

Mice were assigned in a semi-randomized manner and behavioral tests performed between 6 and 9 weeks. Researchers were blind to genotype and treatment for testing and data collection. To minimize experimenter variability, a single investigator conducted each test. Behavior tasks in R6/2 mice were performed as previously described by Ochaba et al. (2016).

### Q140

Males and females were used except for the running wheel, where only males were used since estrus cycle influences running activity. Genotypes or treatments were unknown to the experimenter. All tests were done during the light phase except for the running wheel, conducted during the dark phase. Behavior tasks in Q140 mice were performed as previously described by Hickey et al. (2008).

### Electrophysiology in R6/2 Brain Slices

R6/2 (line 2810, 150±10 CAG repeats) and NT littermates were used, expressing a phenotype similar to that of the 6494 line used for behavioral experiments (Cummings et al., 2012). Procedures were as described by Andre et al. (2011) with modifications as detailed herein.

### Immunohistochemistry and Electron Microscopy

Male R6/2 mice implanted with hNSCs for 5 weeks (n=3) were anesthetized and perfused with EM fixative (2.5% glutaraldehyde, 0.5% paraformaldehyde, and 0.1% picric acid in 0.1 M phosphate buffer [pH 7.4]). Brains were then collected into EM fixative overnight at 4° C. and washed in PBS until serially sectioning through striatum containing hNSCs (equivalent to +1.4 to +0.14 mm from bregma) (Franklin and Paxinos, 2007) at 60 mm using a vibratome (Leica Microsystems). Pre-embed IHC of striatum using diaminobenzidine (DAB) (Sigma, St Louis, Mo.) and hNSC antibody (Stem121, 1:100; StemCells) tissue processed for EM was as previously described (Spinelli et al., 2014; Walker et al., 2012), and striatum slices were embedded flat between two sheets of ACLAR (Electron Microscopy Sciences, Hatfield, Pa.) overnight in a 60° C. oven to polymerize resin. The area containing hNSCs was microdissected from the embedded slice and superglued onto a block for thin sectioning.

Photographs were taken on a JEOL 1400 transmission electron microscope (JEOL, Peabody, Mass.) of DAB-labeled structures (i.e., hNSC-labeled cells, dendrites) at a final magnification of 346,200 using a digital camera (AMT, Danvers, Mass.). Since the DAB labeling was restricted to the leading edge of the thin-sectioned tissue, only the area showing DAB labeling was photographed.

### Biochemical, Molecular, and Immunohistological Analysis in R6/2 Mice

Mice were euthanized by pentobarbital overdose and perfused with 0.01 M PBS. Striatum and cortex were dissected out of the left hemisphere and flash frozen for RNA, and protein isolated in TRIzol using the manufacturer's procedures (Life Technologies, Grand Island, N.Y.) or homogenized as described below. The other halves were post-fixed in 4% paraformaldehyde, cryoprotected in 30% sucrose, and cut at 40 μm on a sliding vibratome for IHC. Sections were rinsed three times and placed in blocking buffer for 1 hr (PBS, 0.02% Triton X-100, 5% goat serum), and primary antibodies placed in block overnight (ON) at 4° C. Sections were rinsed, incubated for 1 hr in Alexa Fluor secondary antibodies, and mounted using Fluoromount G (Southern Biotechnology). Primary antibodies are listed in Supplemental Experimental Procedures.

### Soluble/Insoluble Fractionation

Striatal tissue was processed as described previously (Ochaba et al., 2016). Antibodies: Anti-HTT (Millipore, #MAB5492; RRID: AB_347723) and anti-ubiquitin (Santa Cruz Biotechnology, #sc-8017; RRID: AB_628423). Quantification of bands was performed using software from the NIH program ImageJ and densitometry application.

### Confocal Microscopy and Quantification

Sections were imaged with Bio-Rad Radiance 2100 confocal system using lambda-strobing mode. Images represent either single confocal z slices or z stacks. All unbiased stereological assessments were performed using StereoInvestigator software (MicroBrightField, Williston, Vt.). An optical fractionator probe was used to estimate mean cell, diffuse aggregate, and inclusion body numbers.

RNA Isolation and Real-Time qPCR

Striata were homogenized in TRIzol (Invitrogen), followed by RNEasy Mini kit (Qiagen). RIN values were >9 for each sample (Agilent Bioanalyzer). RT used oligo(dT) primers and 1 mg of total RNA with the SuperScript III First-Strand Synthesis System (Invitrogen). qPCR was performed as described by Vashishtha et al. (2013).

### Biochemical, Molecular, and Immunohistological Analysis in Q140 Mice

Q140 males were euthanized 6 months post treatment by cervical dislocation (n=7 frozen) or paraformaldehyde perfusion (n=5 IHC).

### IHC

Mice were perfused with 0.1 M PBS and 4% paraformaldehyde. The brains were removed, post-fixed in 4% paraformaldehyde overnight, cryoprotected in 30% sucrose, frozen, and coronal sections cut at 40 μm on a cryostat (Leica CM, 1850). Sections were blocked for 1 hr at room temperature and then primary antibodies used ON. After several washes, sections were incubated in Alexa Fluor secondary antibodies and counterstained with DAPI. IHC for the quantification of HTT aggregates and microglia was performed as described by Menalled et al. (2003) and Watson et al. (2012), respectively.

### HTT-Stained Nuclei and Aggregates

Sections were analyzed with StereoInvestigator 5.00 software (Microbrightfield, Colchester, Vt.) (Hickey et al., 2012a). For the contours of striatum drawn, the software laid down a grid of 200×200 μm, with counting frames of 20×20 μm used for quantification of each type of aggregate per section.

### Quantification of IBA-1-Positive Cells in the Striatum of Q140 Mice

Analysis was conducted using a Leica DM-LB microscope with StereoInvestigator software (MicroBrightField) as described for microglial diameter reflecting activation (Watson et al., 2012). For contours of striatum drawn at 5× magnification, the software laid down a grid of 200×200 μm, with counting frames of 20×20 μm at top left corner allowing for unbiased sampling and quantification.

### Biochemical Analysis for Q140 Mice

Frozen striatum processing for ELISAs was performed using a Biosensis BDNF Rapid kit (Biosensis BEK-2211, SA, Australia) as per manufacturer's instructions.

### Statistical Analysis

Results for R6/2 mice are from a single cohort except for the electrophysiology and EM data, which were from a different subset; all used the same batch of cells. Numbers were determined to have sufficient power using an analysis prior to the study (described above). Statistical significance was achieved as described using rigorous analysis. All findings are reproducible. Multiple statistical methods are further detailed above, in figure legends. Significance levels: *p<0.05, **p<0.01, ***p<0.001, ****p<0.0001. In R6/2 mice, data are expressed as mean±SEM; statistical tests for behavior tasks used one-way ANOVA followed by Tukey's HSD test with Scheffe', Bonferroni, and Holm multiple comparison post hoc. Data met the assumptions of the statistical tests used, and p values less than 0.05 were considered significant. All mice were randomly assigned and tasks performed in a random manner with individuals blinded to genotypes and treatment. Statistical comparisons of densitometry results were performed by one-way ANOVA followed by Bonferroni's multiple comparison test. Student's t tests were used for aggregate number comparisons from the EM48 stereological study. Significance in clasping was determined by Fisher's exact probability. Statistical analyses for Q140 mice were conducted using GraphPad Prism 6.0 (GraphPad Software, San Diego, Calif.) for significant differences (p<0.05) in behavioral and postmortem data using one-way ANOVA with Bonferroni post hoc tests. Two-way ANOVA followed by Bonferroni post hoc test was used in the graph representing mean turns in the running wheel/3 min test; and bootstrap statistics using custom MATLAB functions were used for IBA-1 analysis. All error bars on graphs represent SEM.

hNSC isolation. Daily (D) culturing was as follows. D1: ESC colonies enzymatically “loosened” using Collagenase IV until colony edges began lifting. Colonies were manually scraped from wells, transferred to low attachment plates and cultured in EB medium (ESC medium minus bFGF) overnight. D2: EB culture media was supplemented with 500 ng/ml Noggin plus 10 μM SB431542 and cultured for 2 more days. D4: Media change. D5: EBs plated onto growth factor reduced matrigel coated 6-well plates in same medium. D6: Media changed and NP medium used to drive NPC differentiation. Media is changed every two days until the twelfth day. D12-14: Rosettes visually isolated under dissecting scope, manually dissected out using an 18 gauge needle, and plated into growth factor reduced matrigel coated 6-well plates in NSC medium. 2-3 days later, rosettes were dissociated using accutase and plated onto polyornithine/laminin coated plates with NSC medium plus Y27632 compound. ESI-017 hNSC cytogenetic analysis found cells to be karyotypically stable with no observed abnormalities and single color flow-cytometry was performed for CD271 (Brilliant Violet 510—BD Horizon cat #563451), CD24 (Brilliant Violet 711—BD Horizon cat #563401), Pax6 (PE—BD Pharmingen cat #561552), Nestin (Alexa Fluor 647—BD Pharmingen cat #560341), SOX1 (PerCP CY5.5—BD Pharmingen cat #561549), SOX2 (V450—BD Horizon cat #561610), CD44 (APC-H7—BD Pharmingen cat #560532), CD184 (PE-CY7—Biolegend cat #306514) or SSEA4 (lexa Fluor 700—Invitrogen cat #SSEA429).

Transplantation Surgery. Bilateral intra-striatal injections of hNSCs or vehicle were performed using a stereotaxic apparatus and the following coordinates relative to Bregma AP: 0.00 ML: +/−2.00, and DV-3.25. Mice were placed in the stereotaxic frame and injected with either 100,000 hNSCs per side (2 μl/injection) or vehicle (2 μl HBSS with 20 ng/ml hEGF and hFGF) as a control treatment using a 5 μl Hamilton microsyringe (33-gauge) and an injection rate of 0.5 μl/min. R6/2 mice were anesthetized with isoflurane, Q140 mice were anesthetized with sodium pentobarbital (60 mg/kg Nembutal in sterile 0.9% saline, i.p.). For all mice; to maintain a surgical plane of anesthesia mice were administered isoflurane (1-2% in 100% oxygen, 0.5 L/min) via a nose cone, oxygen was administered throughout surgery and 15 temperature of mice was maintained on an electronically controlled heat pad and monitored using a rectal probe thermometer (Physitemp). Accurate placement of the injection to the targeted region was confirmed for all animals by visualization of the needle tract within brain sections. Wounds were sealed using bone wax on the skull and closed with dermabond or with sutures. Mice were placed on heating pads in individual cages after surgery until they recovered from anesthesia. Single daily doses of the immunosuppressant CSA were administered i.p. at a concentration of 10 mg/kg beginning the day before surgeries to hNSC and vehicle implanted R6/2 and non-transgenic mice. To further immunosuppress the mice an additional regimen of i.p. weekly doses of a CD4 antibody (BioXcell, Lebanon, N.H.) was given at 10 mg/kg. Q140 mice or Wt littermates received immunosuppression by CSA (2 mg/kg/day) administered by subcutaneous osmotic minipumps (Alzet #1004) that were changed monthly to ensure the continuous delivery of CSA during the entire study. Surgery to remove and replace minipumps was as follows. Mice were anesthetized with isoflurane (3% for induction and 1.75% for maintenance of anesthesia, in 100% oxygen). After sterilizing the incision site, the minipump was removed through a small incision in the back and new minipumps were implanted before the incision was sutured.

R6/2: Mice were assigned to groups in a semi-randomized manner. The behavioral tests listed below were performed at 6, 7, 8, or 9 weeks of age depending on the task. Mice were weighed daily and no significant differences were observed with treatment. Researchers were blind to which mice had been hNSC transplanted during experiment testing and data collection. To minimize experimenter variability a single investigator conducted each behavioral test. Mice were obtained from breeding colonies at UCI using ovarian transplant female mice (Jackson labs).

The rotarod apparatus was used to measure fore and hind limb motor coordination and balance and mice were tested over 3 consecutive days using an accelerating assay for 300s. The rotarod test was performed every other week two times at ages 6 and 8 weeks. For the pole test mice were placed on the pole with their head pointing down and they then descended head first down the length of the pole. The 16 total time to descend from the starting point of placement was measured. The pole test was performed every other week two times at ages 7 and 9 weeks. An IITC Life Science instrument was used to measure the forelimb grip force via a digital force transducer, the unit gives readings in one gram increments. Grip was measured every other week two times at ages 7 and 9 weeks.

Q140: Climbing test and Pole test. To assess motor coordination and spontaneous activity during climbing mice were placed in the bottom of wire cylinder cages and spontaneous activity was videotaped. For pole test each mouse was positioned face-up at the top of the pole and timed to make a full body-turn into a downward position and timed to descend down the pole into its respective home cage.

### Electrophysiology in R6/2 Mice

Briefly, mice were anesthetized, transcardially perfused with high sucrose-based slicing solution then coronal slices (300 μm) transferred to incubating chamber containing ACSF. MSNs and NSCs were visualized using infrared illumination with differential interference contrast optics. All recordings were performed in or around the injection site (recorded MSNs were adjacent to the graft between 150-200 μm). Biocytin was added to the patch pipette for cell visualization. Spontaneous postsynaptic currents were recorded in the whole-cell configuration in standard ACSF. Membrane currents were recorded in gap-free mode. Cells were voltage-clamped at +10 mV and spontaneous inhibitory postsynaptic currents (sIPSCs) were recorded in ACSF. Spontaneous excitatory postsynaptic currents (sEPSCs) were recorded in ACSF at −70 mV (baseline) and in the presence of the GABAA receptor blocker, bicuculline methobromide (Tocris, Minneapolis, Minn.) to isolate glutamatergic excitatory events. Spontaneous synaptic currents were analyzed using the MiniAnalysis software (version 6.0, Synaptosoft, Fort Lee, N.J.). Following recordings, slices were fixed then transferred to 30% sucrose at 4° C. until IHC processing. To identify biocytin-filled recorded cells and hNSCs, fixed slices were washed, permeabilized and blocked for 4 h, followed by incubation with SC121 (1:1000, StemCells, Inc.). After washing, slices were incubated in goat, antimouse Alexa-488 (1:1000, Life Technologies, Carlsbad, Calif. Catalog #:A-11001) and streptavidinconjugated with Alexa-594 (1:1000, Life Technologies Catalog #: S11227). Slices were washed, mounted and cells visualized with a Zeiss LSM510 confocal microscope.

### Biochemical, Molecular and Immunohistological Analysis in R6/2 Mice.

Confocal Microscopy and Quantification. Sections were imaged with a Bio-Rad Radiance 2100 confocal system using lambda-strobing mode. Images represent either single confocal Z-slices or Zstacks. All unbiased stereological assessments were performed using StereoInvestigator software (MicroBrightField, Williston, Vt.). An optical fractionator probe was used to estimate mean cell numbers, diffuse aggregate numbers and inclusion body numbers. Guard zones were set at 3% of measured thickness with a minimum 14 μm optical dissector height. Contour Tracing was done at 5× objective and counting was performed at 100× objective. For each section, tracing was done approximately 70 μm away from the edges of the stem cell patches. Counting was done in every 3rd section (40 μm coronal sections) for 6 sections throughout the striatum where Ku80 labeled cells could be seen between bregma 0.5 mm and Bregma −0.34 mm. All counts were performed using a 50×50 μm counting frame and 250×250 μm sampling grid in only one brain hemisphere. The CEs value for each Individual mouse ranged between 0.03 and 0.06. Sections were stained for Ku80 using ABC kit and DAB substrate kit (Vector Laboratories) with nickel first, then for EM48 using only ABC and DAB kits. Sections were stained with cresyl violet for non-stem cell nuclear staining. Identical stereological parameters were used to count aggregates and cells on mice implanted with vehicle. Using this stereological assessment of Ku80 positive cells in implanted R6/2 brain sections, ESI-017 NSC implant survival numbers showed an average of 63,975 cells in male mice (n=3) and 18,673 cells in female mice (n=3), equivalent to 64% (males) and 18.6% (females) of initially transplanted cells. There is an overall average of 41,323 cells in mice (n=6, 3 males and 3 females) equivalent to ˜41% of initially transplanted cells. The difference between males and females in number of implanted cells may be due to technical difficulties implanting the smaller females at 5 weeks.

Primary antibodies used for IHC; GFAP (Abcam ab4674), NeuN (Millipore MAB377), SC121 (STEM 121 a human specific cytosolic marker, Clontech AB-121-U-050), Ku80 (Abcam, Cambridge, United Kingdom ab80592), Doublecortin (Millipore AB2253), Olig2 (R&D Systems AF2418), BIIItubulin (Abcam ab107216), MAP-2, (Abcam ab5392), BDNF (Icosagen, 329-100) and EM48 (Millipore MAB5374).

RNA Isolation and Real-Time Quantitative PCR. Brain tissues were homogenized in TRIzol (Invitrogen), and total RNA was isolated using RNEasy Mini kit (QIAGEN). DNase treatment was incorporated into the RNEasy procedure in order to remove residual DNA. RIN values were >9 for each sample (Agilent Bioanalyzer). Reverse transcription was performed using oligo (dT) primers and 1 μg of total RNA using SuperScript III First-Strand Synthesis System (Invitrogen). Quantitative PCR (qPCR) was performed as previously described (Vashishtha, Ng et al. 2013) and ddCT values were quantitated and analyzed against RPLPO. The primers used for amplifying R6/2-Htt transgene were: oIMR1594: 5′-CCCCTCAGGTTCTGCTTTTA-3′, oIMR1596: 5′-TGGAAGGACTTGAGGGACTC-3′; RPLPO Forward: 5′-TGGTCATCCAGCAGGTGTTCGA-3′, RPLPO Reverse: 5′-ACAGACACTGGCAACATTGCGG-3′. Other primers used were Nestin, F 5′TCAAGATGTCCCTCAGCCTGGA3′ R 5′AAGCTGAGGGAAGTCTTGGAGC3′ BDNF F 5′TATGCGCCGAAGCAAGTCTCCA3′ R 5′CATCCAAGGACAGAGGCAGGTA3′ and DCX As 5′GTAAAGCCAACCCTGTGTCG3'S 5′TCCGCTCCAAAATCTGACTC3′.

### Immunohistological Analysis in the Q140 Mice

Primary antibodies used for IHC: HNA (Millipore MAB1281), DCX (abeam ab18723), GFAP (Dako Z033401), or synaptophysin (Millipore 04-1019). IHC for the quantification of HTT aggregates used monoclonal antibody EM48 (Millipore MAB5374) as described (Menalled et al., 2003) and microglia used rabbit anti-Iba-1 (Wako 019-19741) as described (Watson et al., 2012). For cell counts, HNA+ cells were counted over the entire striatal area in 6 coronal sections. 2100 HNA-labeled cells were 19 quantified and the proportion of those cells that were double-labeled with neuronal (DCX, Abeam ab18723) or glial markers (GFAP, Dako Z033401). The final numbers were expressed as the mean of 5 mice per group ±SEM

ESI-017 hNSCs Modify Behavior, Survive, and Differentiate when Transplanted into R6/2 Mice

To evaluate efficacy of hNSC transplantation in a transgenic model of HD, Applicants used exon-1 HTT R6/2 mice (rv120 CAG repeats) (Cummings et al., 2012), which display rapidly progressing motor and metabolic deficits and early death (rv12-14 weeks) (Mangiarini et al., 1996), and can provide an initial assessment of treatment paradigms in preclinical studies (Hickey and Chesselet, 2003; Hockly et al., 2003). ESI-017 hNSCs Improve Behavior

A diagram of the manufacturing process and quality control for the GMP-grade hNSC line is described in FIGS. 8A and 8B. Flow cytometry indicated appropriate staining for hNSC proliferation and pluripotency markers (FIG. 8A). Immunocytochemistry confirmed staining for the neural ectodermal stem cell marker Nestin (FIG. 8C). ESI-017 hNSCs were acquired as frozen aliquots (UC Davis), thawed, and cultured without passaging using the same media reagents as the GMP facility prior to dosing. Five-week-old mice were dosed by intrastriatal stereotactic delivery of 100,000 hNSCs per hemisphere. Male (M) and female (F) R6/2 and non-transgenic (NT) age-matched littermates and vehicle controls (veh) were included (n=8 R6/2 hNSC M, 6 R6/2 hNSC F, 7 NT hNSC M, 7 NT hNSC F, 7 R6/2 veh M, 6 R6/2 veh F, 8 NT veh M, and 6 NT veh F). Immunosuppression was administered to all mice. Behavioral analysis was performed and mice were euthanized at age 9 weeks, immediately following behavior testing.

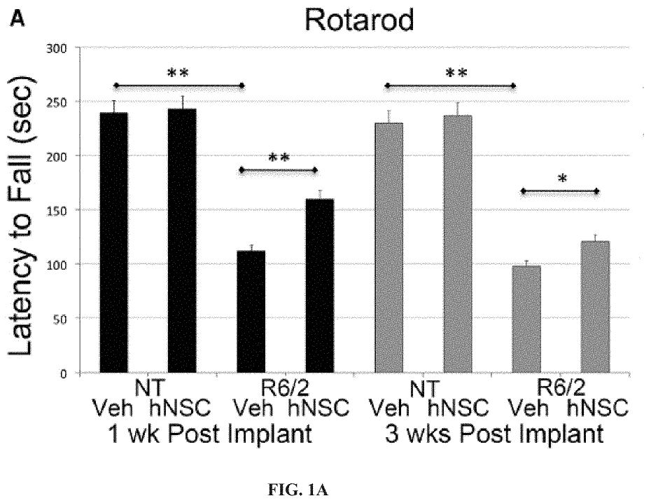

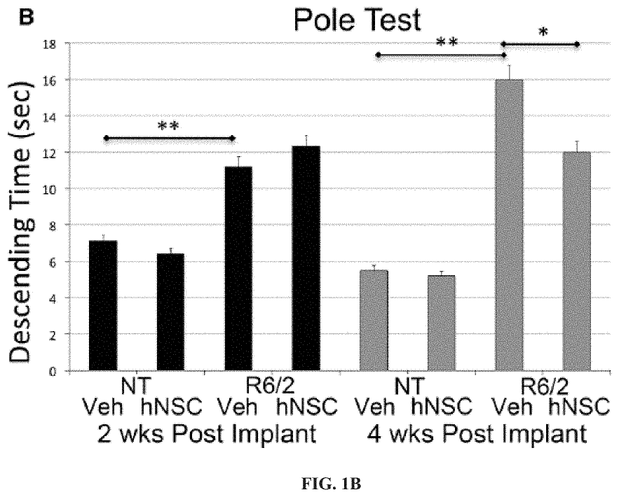

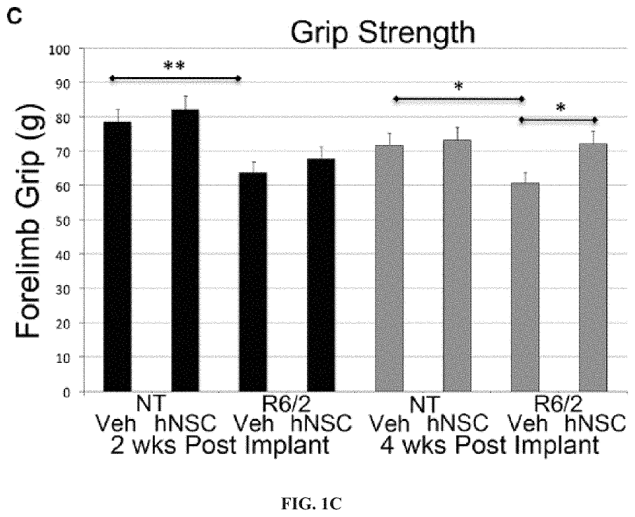

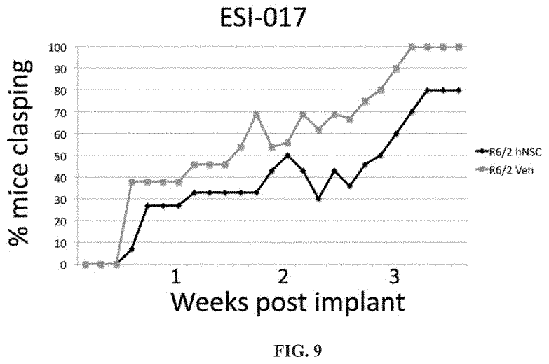

Veh-treated mice developed HD-associated behaviors as described previously (Mangiarini et al., 1996). In brief, behavior of R6/2 mice was indistinguishable from that of NT mice at age 5 weeks. By 8 weeks, neurological abnormalities included progressive stereotypical hind limb grooming movements, clasping, and an irregular gait. When lifted by the tail normal mice splay both hind and forelimbs, and if mice clench limbs to their abdomen they are considered to “clasp.” A delay in onset of R6/2 clasping was observed in all hNSC-treated mice; veh treated mice clasped by 3 weeks post implant. No hNSC treated mice clasped at this time point, and at euthanasia (4 weeks post implant) only 50% of hNSC-treated mice clasped (FIG. 9). Two locomotor assays were performed. Rotarod tests the ability to walk on an accelerating rotating rod. hNSC-treated R6/2 mice showed a statistically significant improvement in Rotarod performance (30% improvement 1 week post implant, p <0.01; and 19% 3 weeks post implant, p<0.05) over veh-treated R6/2 mice (FIG. 1A). The pole test compares times while descending on a vertical beam; R6/2 mice have a longer latency to descend compared with NT mice. A statistically significant (p=0.02) improvement between R6/2 treatment groups was observed at 4 weeks post implant (25% improvement, FIG. 1B). A grip strength meter was also used to assess neuromuscular function and motor coordination, and hNSC treatment produced a significant improvement (p=0.02, 16% improvement, FIG. 1C) 4 weeks post implant.

ESI-017 hNSC Survival, Migration, and Differentiation

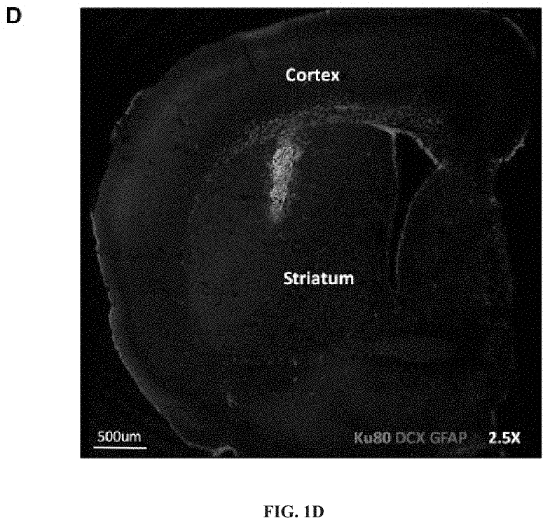

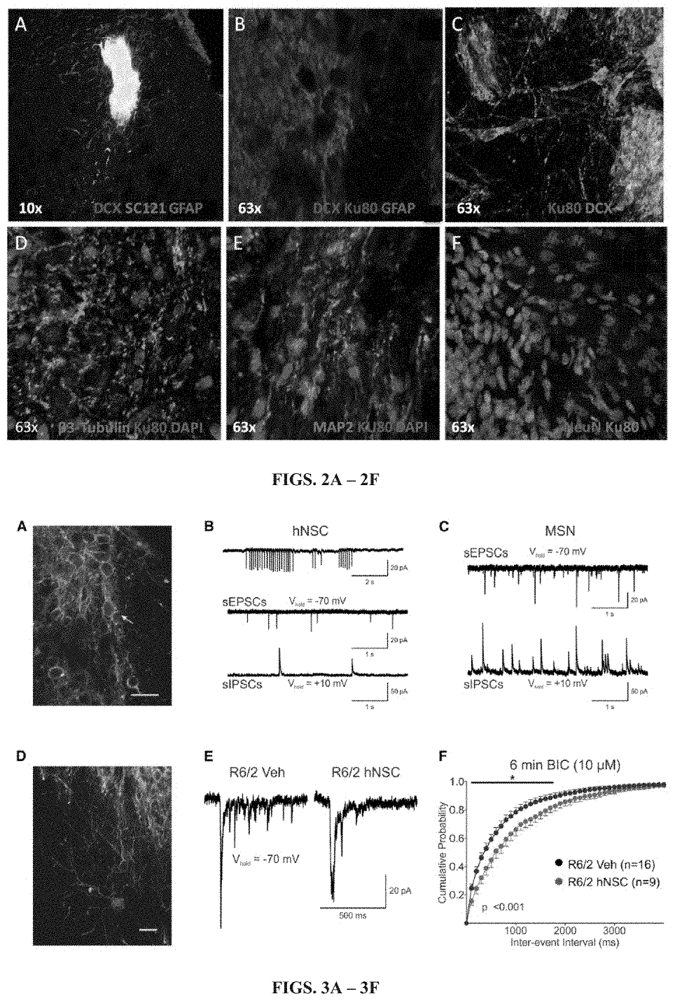

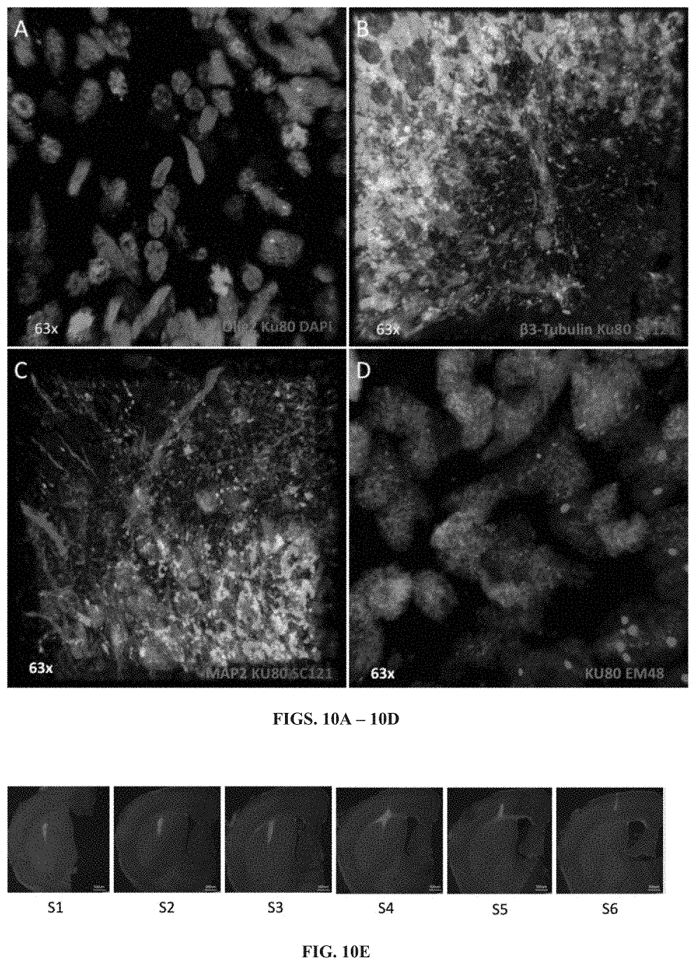

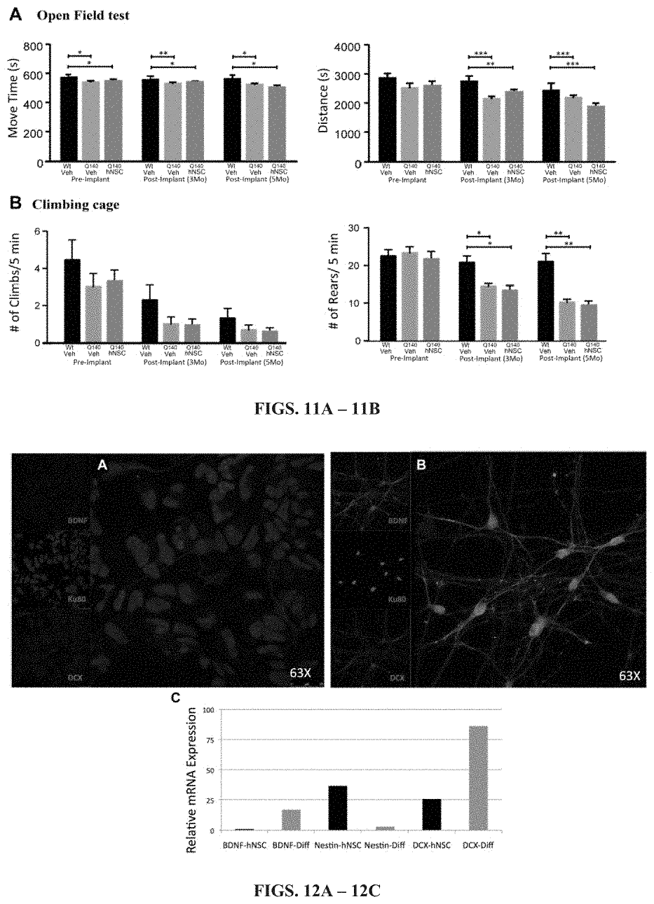

Mice were euthanized 4 weeks post implant and the brain collected, half of which was post-fixed for histology and half flash frozen for biochemistry. hNSCs primarily clustered around the needle track and remained in the striatum (FIG. 1D); some were in the cortex and a few migrated to a niche (corpus callosum/white matter tracts) between the cortical and striatal region (FIG. 10). Using human markers SC121 (cytosolic) or Ku80 (nuclear), cells mainly stained with the early neuronal marker doublecortin (DCX) (SC121, FIG. 2A merge yellow; Ku80, FIGS. 2B and 2C). Some cells potentially differentiated toward an astrocytic phenotype (glial fibrillary acidic protein [GFAP]) (FIG. 2B). There is also non-human GFAP positive immunostaining around the implantation site (FIGS. 2A and 2B) that potentially represents a mouse glial cell scar. The differentiation of hNSCs to neuron restricted progenitors was confirmed with βIII-tubulin (FIGS. 2D and 10B) and microtubule-associated protein 2 (MAP-2) (FIGS. 2E and 10C), but a lack of co-localization with NeuN (FIG. 2F) suggests no post-mitotic neurons. Using stereological assessment of Ku80-positive cells in one hemisphere, hNSC implant survival numbers showed an average of 41,323 cells (n=6, 3 males and 3 females), equivalent to about 41% of the initially transplanted 100,000.

Implantation of ESI-017 hNSCs Prevents Corticostriatal Hyperexcitability in R6/2 Mice

Applicants next evaluated electrophysiological activity. Male and female mice were implanted with 100,000 hNSCs (n=18) or veh (n=16) in striatum at 5 weeks. Applicants recorded from hNSCs 4-6 weeks post implant (FIGS. 3A and 3B) in acute brain slices. hNSCs display basic neuronal properties characteristic of immature cells, a significantly smaller membrane capacitance than host MSNs (hNSC 22.0±1.8 pF, n=31 versus MSN 71.3±3.5 pF, n=44; p<0.001, Student's t test) and a significantly higher membrane input resistance (hNSC 2804.8±203.0 MU versus MSN 163.8±15.1 MU; p<0.001, Student's t test). hNSCs showed spontaneous excitatory and inhibitory postsynaptic currents (sEPSCs and sIPSCs), indicating that they received synaptic inputs from the host tissue or other implanted hNSCs. However, compared with MSNs, frequencies were much lower. Some hNSCs also generated action potentials spontaneously, suggesting that they could affect host neurons and neighboring hNSCs (FIG. 3B).

Electrophysiological alterations occur in MSNs from symptomatic R6/2 compared with NT mice, including changes in intrinsic membrane properties and reduced excitatory synaptic activity (Cepeda et al., 2003, 2007). hNSC implantation did not significantly alter membrane properties, average sEPSC frequency (1.1±0.1 Hz versus 1.4±0.2 Hz) or average SIPSC frequency of MSNs in R6/2 mice. R6/2 mice also display an increase in cortical pyramidal cell excitability and a propensity to develop epileptic discharges and seizures (Cummings et al., 2009). Cortical hyperexcitability is shown in striatal MSNs by the occurrence of large-amplitude EPSCs and high-frequency bursts, particularly evident after extended blockade of GABAA receptors coinciding with an increase in the frequency of sEPSCs (Cepeda et al., 2003; Cummings et al., 2009). A smaller proportion (not statistically significant) of MSNs exhibited increased corticostriatal excitability in hNSC-implanted mice (20.5%, 9/44) compared with veh mice (28.6%, 16/56). However, the increase in sEPSC frequencies within this population did not occur in the R6/2 mice implanted with hNSCs. A rightward shift in the cumulative probability distribution of the inter-event interval plot occurred (p<0.001), indicating that the hNSCs can reduce hyperexcitable input from cortex to striatum when GABAA receptors are blocked (FIGS. 3E and 3F).

Host Tissue Makes Potential Synaptic Contacts with Implanted ESI-017 hNSCs in R6/2 Mice

Applicants utilized immunohistochemistry (IHC) and electron microscopy (EM) to examine whether nerve terminals from the host make synaptic contact with the hNSCs. Applicants found examples of unlabeled nerve terminals originating from the host making a potential symmetrical synaptic contact with the implanted and immunolabeled hNSCs (FIG. 4A). A few synaptic vesicles within the nerve terminal are very close to the presynaptic membrane, indicating a potential area of vesicular release (DAB labeling of hNSCs is obscuring contact). In addition, Applicants found unlabeled nerve terminals originating from the host making a clearly asymmetrical contact (FIG. 4B), suggesting an excitatory synaptic contact. Overall, Applicants found that of the unlabeled nerve terminals originating from the host, 44.5% (n=71) were making an asymmetrical contact while 48.3% (n=69) were making symmetrical contacts with the labeled hNSCs. Of the remaining 7.2% (n=11) of unlabeled nerve terminals originating from the host juxtaposed to the labeled hNSCs, the exact nature of their contact (asymmetrical versus symmetrical) could not be determined.

ESI-017 hNSCs Rescue Behavior, Survive, and Differentiate in Q140 Knockin Mice

Applicants next determined whether hNSCs could also improve deficits in a slowly progressing full-length HD mouse model. Q140 mice express a modified mouse/human exon 1 with 140 repeats inserted into the mouse huntingtin gene (Menalled et al., 2003). Homozygous mice exhibit early abnormalities in motor tests with climbing deficits at age 1.5 months, and cognitive deficits (Hickey et al., 2008; Simmons et al., 2009) and visible aggregates of HTT around 4 months (Menalled et al., 2003). Striatal atrophy is detected at 1 year with a 35% striatal cell loss at 22 months (Hickey et al., 2008). Twenty-four 2-month-old homozygous male and female mice per group were dosed with 100,000 hNSCs per hemisphere, stereotactically delivered bilaterally into the striatum (n=12/sex) with control age-matched Q140 (n=12/sex) and wild-type (WT) (n=12/sex) mice injected with veh. All mice were immunosuppressed. Behavior testing began at age 1.5 months (before cell transplantation) and mice were euthanized at 8 months, 6 months after transplantation. Behavioral tests were performed on all mice except for the running wheel, where only males were used since estrus cycle influences running activity (Hickey et al., 2008). Early deficits in locomotor activity in the open field as well as decrease in spontaneous motor activity in the climbing cage test were observed in Q140 mice; however, hNSC treatment did not rescue performance (FIG. 11).

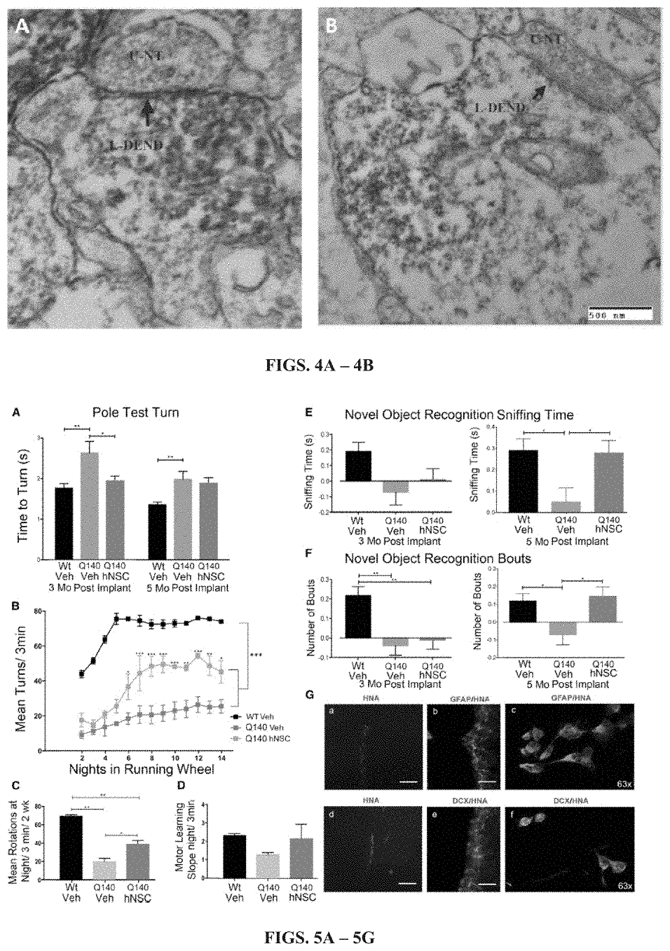

In pole tests veh-treated Q140 mice took longer to turn compared with WT controls (p=0.004); in contrast, hNSC treated Q140 mice were significantly better than control Q140 mice (p=0.04) and no longer significantly different from WT, indicating a beneficial effect 3 months post transplantation (FIG. 5A). As reported by Hickey et al. (2008), 5.5-month-old male Q140 mice had profound deficits in running speed (rotations per 3 min), significant for 2 weeks (FIG. 5B). Persistent improvement of running wheel deficits was observed post treatment with hNSC-treated Q140 mice, showing a progressive increase in average running wheel activity compared with veh-treated mice (FIGS. 5B and 5C). Applicants concluded that hNSC administration improved some of the motor deficits observed in Q140 mice. Novel object recognition (NOR) is a cortical-dependent cognitive test that requires both learning and memory (recognition) and takes advantage of the tendency of mice to investigate a novel object over a familiar one. Veh-injected Q140 mice exhibited significant impairments in NOR compared with veh-injected WT mice at 3 and 5 months post implant (p=0.003 and p=0.03, respectively) as reported by Simmons et al. (2009). Striatal transplantation of hNSCs in Q140 mice rescued cognitive impairments at 5 months post implant (p=0.03), but not earlier (FIGS. 5E and 5F).

A subset (n=5 for each group) of veh- and hNSC-transplanted Q140 male mice were euthanized at 6 months post treatment for IHC analyses. hNSCs, identified with a human nuclear-specific antibody (HNA), were present 6 months post transplantation and mostly confined to the injection tract (FIG. 5Ga,b) in the striatum. The number of HNA-positive cells counted over the entire striatal area in six coronal sections and cells double-labeled with DCX or GFAP was calculated (mean data from 5 mice per group ±SEM). Approximately 25% of the 100,000 hNSCs survive with most (84%±2%) being GFAP positive (FIG. 5Gb,c), a smaller proportion (16%±2%) being DCX positive (FIG. 5Ge,f).

ESI-017 hNSC Transplantation Increases BDNF Levels in HD Mice

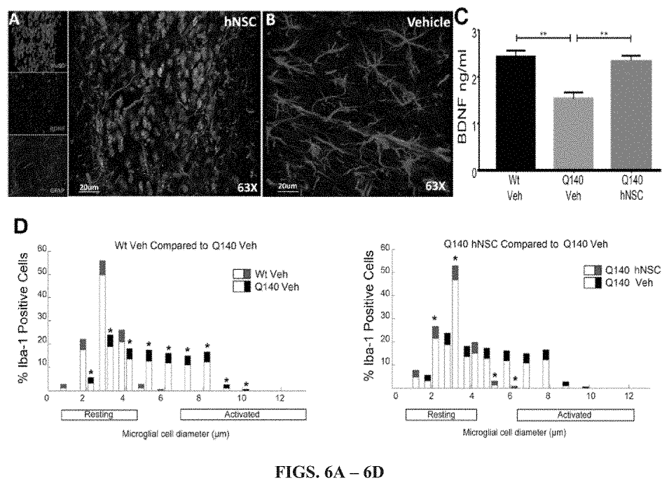

Increased levels of neurotrophic growth factors and subsequent increased synaptic connectivity are implicated in behavioral ameliorations observed after transplantation of NSCs (Blurton-Jones et al., 2009). Furthermore, reduced BDNF has been demonstrated for multiple mouse models of HD and in human HD brain (Zuccato et al., 2011). Therefore, we evaluated BDNF levels as a marker for neurotrophic effects. In the R6/2 hNSC mice, IHC and confocal microscopy indicated co-localization of BDNF with DCX-positive hNSCs, suggesting that the differentiated cells produce BDNF (FIG. 6A). Indeed, hNSCs grown in vitro and differentiated produce BDNF only after becoming DCX positive. In the Q140 hNSC mice, BDNF was quantified by ELISA in a subset of male mice (n=6/group). Striatal BDNF was decreased in Q140 mice compared with WT, but a significant increase in BDNF levels was observed in hNSC-treated compared with veh, restoring it to WT levels (FIG. 6C).

Given that neurotrophic signaling can enhance synaptic activity, we examined levels of synaptophysin, a synaptic marker, in the striatum of all perfused Q140 animals (n=5/group) by IHC and quantification using a microarray scanner as previously described (Richter et al., 2017). Comparison of hNSC—with veh-treated Q140 mice revealed a significant increase in synaptophysin in the hNSC mice (FIG. 13A, quantified in FIG. 13B).

These results suggest that engrafted hNSCs may in part improve synaptic connectivity by increased neurotrophic effects, including BDNF.

ESI-017 hNSC Treatment in Q140 Mice Decreased Microglial Activation

Striatal sections from Q140 mice (n=5/group) were stained with an Ionized calcium-binding adaptor molecule 1 (Iba-1) antibody which identifies both resting and reactive microglia. Microglial soma sizes correlate with activation state cell morphology (Watson et al., 2012) and a significant increase in the diameter of Iba1-positive cells (strong microglial response) was observed in the striatum of Q140 mice. This response was significantly reduced by hNSCs (FIG. 6D). Similar analysis in hNSC-implanted R6/2 mice did not show a significant alteration in the striatum (FIG. 13) and may be due to a relatively localized effect or a moderate level of activated microglia.

ESI-017 hNSC Transplantation Reduces mHTT Accumulation and Aggregates

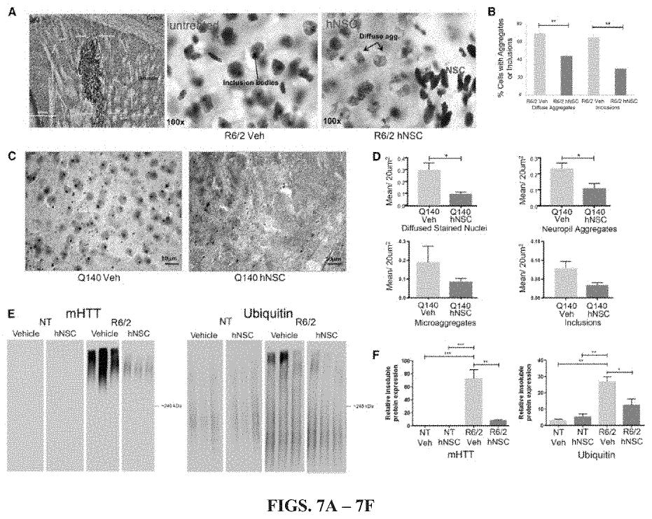

A hallmark of HD pathology is the presence of HTT inclusions that may reflect altered protein homeostasis. Therefore, we performed unbiased stereological assessments on brain sections from R6/2 and Q140 mice. For R6/2 mice, sections were stained first for Ku80 with nickel-enhanced DAB (black), then for HTT (EM48) using DAB without nickel, then with cresyl violet counterstain for non-hNSC nuclear staining. FIG. 7A shows the area where stereology was performed adjacent to the hNSC implant; areas away from the implant did not show significant differences in mutant HTT (mHTT) accumulation or aggregates. Results indicate that R6/2 mice implanted with hNSCs have decreased diffuse staining and decreased inclusion numbers near the injection site compared with veh (FIGS. 7A and 7B).

A clear decrease in aggregate numbers was also observed in the striatum of Q140 mice (FIG. 7C). At 6 months post treatment, hNSC-treated Q140 mice had fewer diffusely stained nuclei (p=0.0102) and fewer neuropil aggregates (p=0.0239), but no reduction in nuclear inclusions nor microaggregates (p=0.0753 and p=0.372, respectively) compared with veh treated mice (FIG. 7D). This result suggests that hNSC delivery modulated HD-related pathology. No acquisition of inclusions was observed in or near transplanted cells in either R6/2 (FIG. 10D) or Q140 mice.

hNSC Transplantation Decreases Pathogenic Accumulation of mHTT and Ubiquitinated Proteins

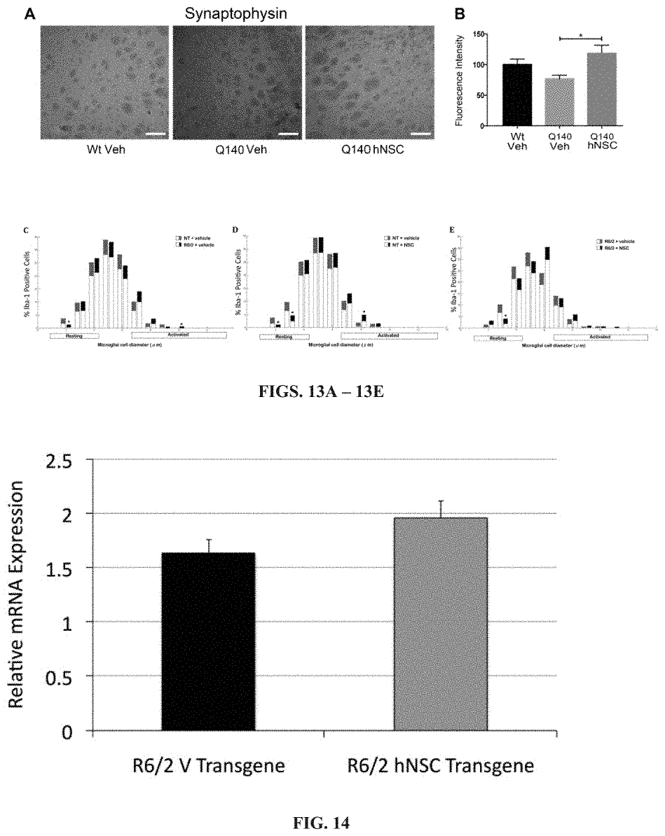

Applicants next examined the impact of hNSC treatment on high molecular weight (HMW) mHTT species and ubiquitin modified proteins that accumulate in R6/2 brain. Reduction of these insoluble proteins corresponds to improved behavioral outcomes in R6/2 mice (Ochaba et al., 2016). Evaluation of a detergent-insoluble fraction of NT and R6/2 striatum with and without hNSC transplantation indicated that accumulated mHTT levels were significantly increased in R6/2 striatum, and treatment with hNSCs decreased insoluble HTT accumulation by about 70% in R6/2 striatum compared with veh-treated mice (FIGS. 7E and 7F), which was not due to altered mHTT transgene mRNA expression (FIG. 14). Accumulated ubiquitin-conjugated proteins were also significantly increased in R6/2 striatum compared with NT mice and hNSC treatment decreased insoluble ubiquitin-conjugated proteins in R6/2 mouse striatum compared with veh-treated mice (FIGS. 7E and 7F). No significant difference was detected in treated NT mice.

The CCT/TRiC (TCP1-ring complex) chaperonin is an oligomeric chaperone that binds and folds newly translated polypeptides. CCT/TRiC expression prevents truncated mHTT aggregation in multiple HD model systems (Tam, S., et al., The chaperonin TRiC controls polyglutamine aggregation and toxicity through subunit-specific interactions. Nature Cell Biol, 2006. 8(10): p. 1155-1162). Over-expression of one subunit, CCT1, is sufficient to inhibit aggregation in vitro and in cells, and reduce mHTT-mediated cell toxicity (Tam, S., et al., The chaperonin TRiC blocks a huntingtin sequence element that promotes the conformational switch to aggregation. 2009. 16(12): p. 1279-1285). Strikingly, the 20 kDa apical domain of yeast CCT1 (ApiCCT1) is sufficient to inhibit aggregation of recombinant mHTT in vitro. Applicants' data show that recombinant ApiCCT1, ApiCCT1r, can reduce HD phenotypes in cells (Sontag, E. M., et al., Exogenous delivery of chaperonin subunit fragment ApiCCT1 modulates mutant Huntingtin cellular phenotypes. Proc Natl Acad Sci U S A, 2013. 110(8): p. 3077-82) and rescue BDNF trafficking deficits in co-cultures of HD mouse primary neurons (Zhao, X., et al., TRiC subunits enhance BDNF axonal transport and rescue striatal atrophy in Huntington's disease. Proc Natl Acad Sci USA, 2016). Importantly, this exogenously applied ApiCCT1r is taken up into the cytosol of cultured cells and primary neurons to exert effects (Sontag et al, Zhao et al), suggesting that if one can deliver the protein to disease tissue, ApiCCT1 could be taken up by cells and have beneficial effects. A single direct injection of ApiCCT1 into R6/1 striatum was detected even after 2 weeks and reduced levels of high molecular weight and aggregated HTT. In more recent preliminary data, viral-mediated delivery of sApiCCT1 or delivery of mouse NSCs secreting ApiCCT1 provides improvement in vivo in HD mice. These data suggest that continuous delivery of ApiCCT1 could be neuroprotective.

### Viral-Mediated Delivery of ApiCCT1 is Efficacious In Vivo.

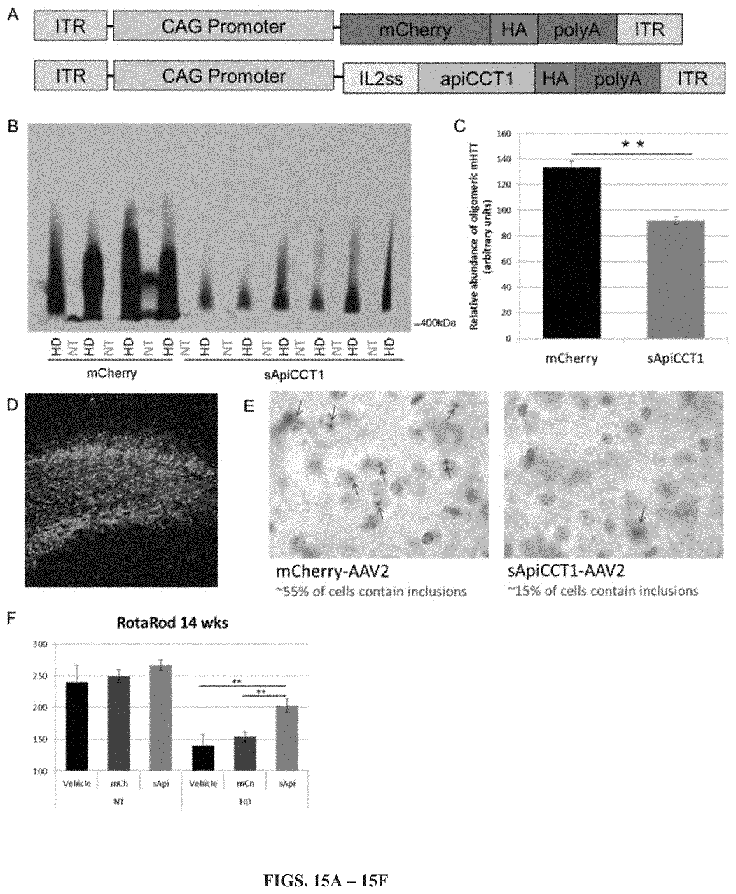

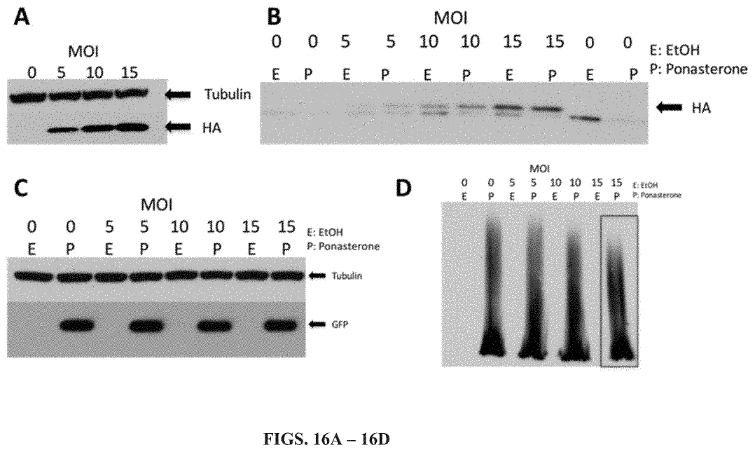

To assess continuous sApiCCT1 delivery in vivo, AAV2/1-mediated delivery of sApiCCT1 was tested in a small pilot study for its effect on mHTT accumulation in R6/1 mice, expressing exon 1 of human mHTT with ˜115 repeats and displaying a slower course of disease progression than R6/2 [24] (Constructs in FIG. 15A). Because of the rapid onset of phenotypes in R6/2 mice and the 2-3 weeks for AAV2/1 to reach full expression, delivery of virus earlier in disease progression may be essential to achieve maximum correction of pathological phenotypes. Bilateral intrastriatal injections (12×10⁹ genome copies of AAV2/1 expressing sApiCCT1 or mCherry control) to R6/1 mice were therefore performed at 5 weeks of age and tissues harvested at 17 weeks of age. Animals injected with sApiCCT1 showed an approximate 40% reduction in oligomeric mHTT (FIG. 15B&C). Analysis by stereology also revealed an approximate 40% reduction in visible inclusions (FIG. 15E), although this effect was not statistically significant presumably due to an underpowered sample size. These animals displayed a significant improvement in clasping behavior at 16 weeks of age; this assay is an indication of motor impairment (data not shown). The study was repeated with a larger sample size (˜20 each condition). Animals injected with AAV2/1-sApiCCT1 showed significant improvements in rotarod task, which measures motor coordination and balance, at 10, 12, and 14 weeks (FIG. 15F; 10 and 12 week data not shown). These animals also showed improvements in clasping behavior, consistent with the previous study (data not shown). Taken together, these studies suggest that continuous delivery of sApiCCT1 is sufficient to improve behavioral outcomes and reduce mHTT pathology in HD mice.

Viral-Transduced hNSCs Produce Secreted ApiCCT1 that Enters Htt14A2.6 PC12 Cells and Impacts Oligomeric mHTT Species

Applicants have performed a small pilot study to test sApiCCT lentivirus transduction of ESI-017 hNSCs to determine the appropriate titer for transduction and to examine ApiCCT production as well as effects on mutant HTT aggregation. Briefly, ESI-017 hNSCs were cultured in 6 well plates then transduced with sApiCCT lentivirus at Multiplicity of Infection (MOI) of 0, 5, 10 and 15. Cells were cultured for 48 hours, media was collected and cells harvested for protein analysis. FIG. 16A shows Western analysis of HA tagged ApiCCT and indicates the transduced ESI-017 hNSCs are producing ApiCCT and that production increases as virus MOI is increased. Media collected from the transduced hNSCs was added to Htt14A2.6 PC12 cell media to determine that transduced and secreted ApiCCT can enter neighboring cells as previously described (Sontag PNAS, 2013). In the presence of the inducer, ponasterone, these cells express a truncated form of expanded repeat HTT exon 1 protein (103Qs) fused at the C terminus to enhanced green fluorescent protein (GFP) within 48 hours. 48 hours after induction and application of the conditioned media, cells were washed, harvested and Western analysis performed. Results indicate that cell lysates from the treated 14A2.6 cells contain an HA tagged protein of the appropriate molecular weight to be ApiCCT (FIG. 16B). To evaluate if conditioned media delivery of ApiCCT had effects on specific mutant Huntingtin (mHTT) aggregation species, Applicants first evaluated if levels of monomeric, soluble HTT fragment was altered. HTT monomer levels from the same experiments were examined by Western analysis using an antibody to GFP (FIG. 16C). ApiCCT1 does not appear to alter expression of monomeric levels of mHTT, suggesting that ApiCCT does not alter the steady-state levels of monomeric mutant HTT (mHTT) and does not appear to influence gene expression of induced mHTT. Insoluble HTT aggregates and mHTT oligomers are hallmarks of HD. In particular, oligomeric mHTT species may represent a source of toxicity in affected neurons. Therefore, Applicants measured mHTT oligomers to determine whether delivery of ApiCCT1 influences accumulation of these forms as previously demonstrated with direct delivery of purified ApiCCT1 protein. SDS agarose gel electrophoresis (AGE) was used to resolve oligomeric species, as this approach seems to preferentially resolve soluble fibrillar oligomers of mHTT. Equivalent amounts of protein from cell lysates were loaded on SDS-AGE gels. Using ImageJ to obtain densitometry measurements, ApiCCT1 caused a decrease in the level of mHTT oligomers (>10%) only at the highest MOI (FIG. 16D). However, smear length was reduced at both MOI 10 and 15. These data indicate that ApiCCT1 secretion from hNSCs is able to reduce the formation of oligomeric mHTT in neighboring cells, reproducing our published results for purified ApiCCT1. These results validate methods to be employed in GMP production of Lentivirus transduction of hNSCs and establish the potential of hNSC delivery.

Viral-Transduced hNSCs Produce Secreted ApiCCT1 after Implantation into Mice

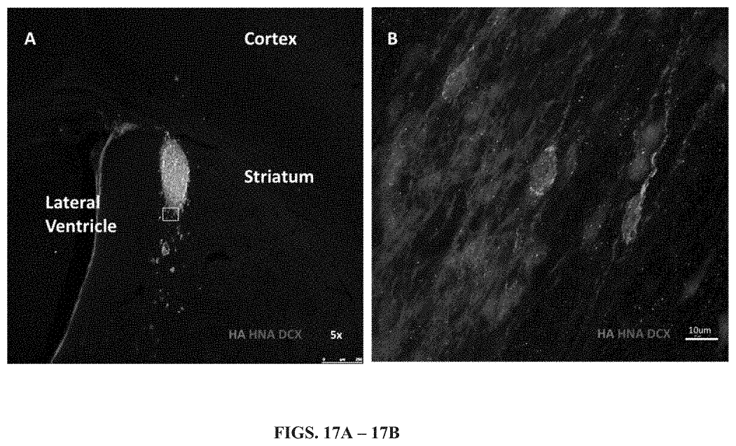

ESI-017 hNSCs were cultured at UCI as described above. hNSCs were transduced with lentivirus at MOI 15 for 48 hrs then transplanted into five-week-old mice as described above. Male and female R6/2 and non-transgenic age-matched littermates and vehicle controls were included. Immunosuppression was administered to all mice. Mice were euthanized at age 9 weeks and the brain collected, half of which was post-fixed for histology and half flash frozen for biochemistry. hNSC-ApiCCT implanted cells had similar IHC as described for hNSCs (FIG. 17). Using human nuclear antigen marker (HNA), cells mainly stained with the early neuronal marker doublecortin (DCX, blue) (FIG. 17A merge pink). Some cells express the HA tagged ApiCCT (FIG. 17B).

### Discussion

Stem cell-based transplantation strategies are promising approaches for neurodegenerative disorders based on their ability to modulate pathology through regenerative and restorative mechanisms. In HD models, mouse-derived NSCs have shown promising results while hNSC-based approaches have had mixed success, with robust efficacy in toxin models and limited neuroprotection in genetic HD mice (El-Akabawy et al., 2012; Golas and Sander, 2016). Here we describe transplantation of GMP-grade hNSCs that provides robust rescue of deficits and disease-modifying activity targeting the accumulation of the mHTT protein. ESI-017 hNSCs were electrophysiologically active in R6/2 mice but did not have significant effects on striatal MSN membrane properties or spontaneous synaptic activity. In a subset of MSNs, however, the increase in frequency of sEPSCs commonly observed after extended blockade of GABAA receptors with bicuculline did not occur, suggesting that the grafts help to reduce cortical hyperexcitability. Applicants have not determined the underlying mechanisms of this effect, but electrical stimulation inside the graft induces IPSCs in neighboring cells, suggesting that they are inhibitory. The ultrastructural data show that the host is potentially making both symmetrical (inhibitory) and asymmetrical (excitatory) synaptic contacts in equal numbers with the hNSCs. Our assumption is that the effects are derived from the implanted cells and that in R6/2 mice they are primarily differentiating along a neuronal lineage. However, in other experiments including the Q140 mice, there is a potential glial effect, suggesting that the driver of improvement is not yet understood. Given that neuronal loss does not occur in these mice until very late stages of disease, the striatal-specific transplantation appears to act through both neuroprotection via trophic factors such as BDNF and by preventing the aberrant accumulation of mHTT species. However, the finding of electrophysiological activity in transplanted cells, and contact between human and endogenous mouse cells that may facilitate improved electrophysiological outcomes, suggest that there may also be an opportunity for regenerative effects.

The rationale for transplanting NSCs versus other progenitor types is based on their ability to differentiate along multiple lineages. In R6/2 mice, cells exhibited evidence of early astrocytic or neuronal differentiation; most co-label with neuron-restricted progenitor markers (DCX, βIII-tubulin, and MAP-2). As hNSCs typically take several months to terminally differentiate, we expected to observe only partial differentiation of transplanted cells at the 4-week time point. Interestingly, very few ESI-017 hNSCs are DCX positive before implantation in vitro. Results of cell fate in R6/2 mice are in contrast to our findings in the Q140 long-term HD model and other studies in Parkinson's disease and Alzheimer's disease (AD) models using hNSCs where more cells are becoming astrocytes (Goldberg et al., 2017), although the latter are derived from fetal NSCs, which tend to be more gliogenic. These data suggest that there may be different responses depending on the disease niche, immunosuppression paradigms may influence specification, or developmental cues and timing specific to human versus mouse cells influences outcomes.

Diminished BDNF levels are present in HD mice and in human HD subjects (Strand et al., 2007; Zuccato et al., 2011), and many efficacious treatments in HD mice show a concomitant increase in BDNF (Ross and Tabrizi, 2011). Consistent with the idea of trophic factor support through stem cell transplantation; ex vivo delivery of mouse NSCs expressing GDNF maintains motor function and prevents neuronal loss in HD mice (Ebert et al., 2010), and BDNF was required for improved cognition following mouse NSC transplantation into either AD mice (Blurton-Jones et al., 2009) or a model of dementia with Lewy bodies (Goldberg et al., 2015). BDNF must be trafficked to the striatum via the afferent pathways, including the corticostriatal pathway that is altered in HD (Laforet et al., 2001). It is possible that by supplying trophic support to the striatum, the corticostriatal pathway is preserved enough to signal BDNF production in the cortex or that stem cell-derived BDNF is retrogradely transported from the striatum back to the soma of corticostriatal neurons, leading to improved electrophysiological activity following transplantation.

One mechanism of action of implanted hNSCs may be via reduction of aberrant mHTT accumulation and aggregates, potentially through preventing their formation or inducing selective clearance mechanisms (e.g., Chen et al., 2013).We recently described findings that reduction of a specific HMW insoluble mHTT species was associated with improved behavior and normalization of several molecular readouts in R6/2 mice (Ochaba et al., 2016). It is plausible that reduction of pathogenic accumulation of mHTT and ubiquitinated HMW insoluble species prevents the neuronal dysfunction that is observed in the HD mice.

It is important to note that in contrast to the observation that aggregates could be acquired in a study of fetal cell transplants in human HD subjects (Cicchetti et al., 2014), no evidence of acquired HD phenotypes, such as inclusions, were observed over the course of the transplants in either mouse model (FIG. 10). The lack of apparent protein propagation or acquired pathology could be a result of increased trophic signaling of the hNSCs or from reducing mHTT species that could otherwise facilitate protein propagation into the transplanted cells. Alternatively, it could take years for the cells to acquire pathology, which is not represented by the mouse studies.

In summary, we show that hNSCs transplanted into HD mice survived, differentiated into neural populations, may protect or repair damaged tissue and delay disease progression, decreased pathologies and increased production of protective molecules, and potentially formed contacts with surrounding tissue, suggesting a prospective treatment strategy for HD. Given the results by An et al. (2012) showing that genetically corrected patient-derived NSCs can form human neurons and DARPP-32-positive cells and the results reported here, future application of autologous transplantation using corrected patient cells may also be feasible.

### EQUIVALENTS

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control. Throughout this specification, technical literature is referenced by an author citation, the complete bibliographic details for which are provided below.

### REFERENCES

1. Ager R. R., Davis J. L., Agazaryan A., Benavente F., Poon W. W., LaFerla F. M., Blurton-Jones M. Human neural stem cells improve cognition and promote synaptic growth in two complementary transgenic models of Alzheimer's disease and neuronal loss. Hippocampus. 2015; 25:813-826. [PubMed]
2. An M. C., Zhang N., Scott G., Montoro D., Wittkop T., Mooney S., Melov S., Ellerby L. M. Genetic correction of Huntington's disease phenotypes in induced pluripotent stem cells. Cell Stem Cell. 2012; 11:253-263. [PubMed]
3. Andre V. M., Cepeda C., Fisher Y. E., Huynh M., Bardakjian N., Singh S., Yang X. W., Levine M. S. Differential electrophysiological changes in striatal output neurons in Huntington's disease. J. Neurosci. 2011; 31:1170-1182. [PubMed]
4. Benraiss A., Wang S., Herrlinger S., Li X., Chandler-Militello D., Mauceri J., Burm H. B., Toner M., Osipovitch M., Jim Xu Q. Human glia can both induce and rescue aspects of disease phenotype in Huntington disease. Nat. Commun. 2016; 7:11758. [PubMed]
5. Blurton-Jones M., Kitazawa M., Martinez-Coria H., Castello N. A., Muller F. J., Loring J. F., Yamasaki T. R., Poon W. W., Green K. N., LaFerla F. M. Neural stem cells improve cognition via BDNF in a transgenic model of Alzheimer disease. Proc. Natl. Acad. Sci. USA. 2009; 106:13594-13599. [PubMed]
6. Cepeda C., Hurst R. S., Calvert C. R., Hernandez-Echeagaray E., Nguyen O. K., Jocoy E., Christian L. J., Ariano M. A., Levine M. S. Transient and progressive electrophysiological alterations in the corticostriatal pathway in a mouse model of Huntington's disease. J. Neurosci. 2003; 23:961-969. [PubMed]
7. Cepeda C., Wu N., Andre V. M., Cummings D. M., Levine M. S. The corticostriatal pathway in Huntington's disease. Prog. Neurobiol. 2007; 81:253-271. [PubMed]
8. Chen A., Xiong L. J., Tong Y., Mao M. Neuroprotective effect of brain-derived neurotrophic factor mediated by autophagy through the PI3K/Akt/mTOR pathway. Mol. Med. Rep. 2013; 8:1011-1016.[PubMed]
9. Cicchetti F Lacroix S Cisbani G Vallieres N Saint-Pierre M St-Amour I., Tolouei R., Skepper J. N., Hauser R. A., Mantovani D. Mutant huntingtin is present in neuronal grafts in Huntington disease patients. Ann. Neurol. 2014; 76:31-42. [PubMed]
10. Cummings D. M., Andre V. M., Uzgil B. O., Gee S. M., Fisher Y. E., Cepeda C., Levine M. S. Alterations in cortical excitation and inhibition in genetic mouse models of Huntington's disease. J. Neurosci. 2009; 29:10371-10386. [PubMed]
11. Cummings D. M., Alaghband Y., Hickey M. A., Joshi P. R., Hong S. C., Zhu C., Ando T. K., Andre V. M., Cepeda C., Watson J. B. A critical window of CAG repeat-length correlates with phenotype severity in the R6/2 mouse model of Huntington's disease. J. Neurophysiol. 2012; 107: 677-691. [PubMed]
12. Drouin-Ouellet J. The potential of alternate sources of cells for neural grafting in Parkinson's and Huntington's disease. Neurodegener. Dis. Manag. 2014; 4:297-307. [PubMed]
13. Ebert A. D., Barber A. E., Heins B. M., Svendsen C. N. Ex vivo delivery of GDNF maintains motor function and prevents neuronal loss in a transgenic mouse model of Huntington's disease. Exp. Neurol. 2010; 224:155-162. [PubMed]
14. El-Akabawy G., Rattray I., Johansson S. M., Gale R., Bates G., Modo M. Implantation of undifferentiated and pre-differentiated human neural stem cells in the R6/2 transgenic mouse model of Huntington's disease. BMC Neurosci. 2012; 13:97. [PubMed]
15. Franklin K., Paxinos G. Third Edition. Academic Press; 2007. The Mouse Brain in Stereotaxic Coordinates.
16. Golas M. M., Sander B. Use of human stem cells in Huntington disease modeling and translational research. Exp. Neurol. 2016; 278:76-90. [PubMed]
17. Goldberg N. R., Caesar J., Park A., Sedgh S., Finogenov G., Masliah E., Davis J., Blurton-Jones M. Neural stem cells rescue cognitive and motor dysfunction in a transgenic model of dementia with lewy bodies through a BDNF-dependent mechanism. Stem Cell Rep. 2015; 5:791-804.[PMC free article] [PubMed]
18. Goldberg N. R., Marsh S. E., Ochaba J., Shelley B. C., Davtyan H., Thompson L. M., Steffan J. S., Svendsen C. N., Blurton-Jones M. Human neural progenitor transplantation rescues behavior and reduces alpha-synuclein in a transgenic model of dementia with Lewy bodies. Stem Cells Transl. Med. 2017; 6:1477-1490. [PubMed]
19. Hickey M. A., Chesselet M. F. The use of transgenic and knock-in mice to study Huntington's disease. Cytogenet. Genome Res. 2003; 100:276-286. [PubMed]
20. Hickey M. A., Gallant K., Gross G. G., Levine M. S., Chesselet M. F. Early behavioral deficits in R6/2 mice suitable for use in preclinical drug testing. Neurobiol. Dis. 2005; 20:1-11. [PubMed]
21. Hickey M. A., Kosmalska A., Enayati J., Cohen R., Zeitlin S., Levine M. S., Chesselet M. F. Extensive early motor and non-motor behavioral deficits are followed by striatal neuronal loss in knock-in Huntington's disease mice. Neuroscience. 2008; 157:280-295. [PubMed]
22. Hickey M. A., Zhu C., Medvedeva V., Franich N. R., Levine M. S., Chesselet M. F. Evidence for behavioral benefits of early dietary supplementation with CoEnzymeQ10 in a slowly progressing mouse model of Huntington's disease. Mol. Cell Neurosci. 2012; 49:149-157. [PubMed]
23. Hickey M. A., Zhu C., Medvedeva V., Lerner R. P., Patassini S., Franich N. R., Maiti P., Frautschy S. A., Zeitlin S., Levine M. S. Improvement of neuropathology and transcriptional deficits in CAG 140 knock-in mice supports a beneficial effect of dietary curcumin in Huntington's disease. Mol. Neurodegener. 2012; 7:12. [PubMed]
24. Hockly E., Woodman B., Mahal A., Lewis C. M., Bates G. Standardization and statistical approaches to therapeutic trials in the R6/2 mouse. Brain Res. Bull. 2003; 61:469-479. [PubMed]
25. Kirkeby A., Parmar M., Barker R. A. Strategies for bringing stem cell-derived dopamine neurons to the clinic: a European approach (STEM-PD) Prog. Brain Res. 2017; 230:165-190. [PubMed]
26. Laforet G. A., Sapp E., Chase K., McIntyre C., Boyce F. M., Campbell M., Cadigan B. A., Warzecki L., Tagle D. A., Reddy P. H. Changes in cortical and striatal neurons predict behavioral and electrophysiological abnormalities in a transgenic murine model of Huntington's disease. J. Neurosci. 2001; 21:9112-9123. [PubMed]
27. Mangiarini L., Sathasivam K., Seller M., Cozens B., Harper A., Hetherington C., Lawton M., Trottier Y., Lehrach H., Davies S. W. Exon 1 of the HD gene with an expanded CAG repeat is sufficient to cause a progressive neurological phenotype in transgenic mice. Cell. 1996; 87:493-506.[PubMed]
28. Menalled L. B., Sison J. D., Dragatsis I., Zeitlin S., Chesselet M. F. Time course of early motor and neuropathological anomalies in a knock-in mouse model of Huntington's disease with 140 CAG repeats. J. Comp. Neurol. 2003; 465:11-26. [PubMed]
29. Mu S., Wang J., Zhou G., Peng W., He Z., Zhao Z., Mo C., Qu J., Zhang J. Transplantation of induced pluripotent stem cells improves functional recovery in Huntington's disease rat model. PLoS One. 2014; 9:e101185. [PubMed]
30. Ochaba J., Monteys A. M., O'Rourke J. G., Reidling J. C., Steffan J. S., Davidson B. L., Thompson L. M. PIAS1 regulates mutant Huntingtin accumulation and Huntington's disease-associated phenotypes in vivo. Neuron. 2016; 90:507-520. [PubMed]
31. Petit G. H., Olsson T. T., Brundin P. The future of cell therapies and brain repair: Parkinson's disease leads the way. Neuropathol. Appl. Neurobiol. 2014; 40:60-70. [PubMed]
32. Pollock K., Dahlenburg H., Nelson H., Fink K. D., Cary W., Hendrix K., Annett G., Torrest A., Deng P., Gutierrez J. Human mesenchymal stem cells genetically engineered to overexpress brain-derived neurotrophic factor improve outcomes in Huntington's disease mouse models. Mol. Ther. 2016; 24:965-977. [PubMed]
33. Richter F., Gabby L., McDowell K. A., Mulligan C. K., De La Rosa K., Sioshansi P. C., Mortazavi F., Cely I., Ackerson L. C., Tsan L. Effects of decreased dopamine transporter levels on nigrostriatal neurons and paraquat/maneb toxicity in mice. Neurobiol. Aging. 2017; 51:54-66. [PubMed]
34. Ross C. A., Tabrizi S. J. Huntington's disease: from molecular pathogenesis to clinical treatment. Lancet Neurol. 2011; 10:83-98. [PubMed]
35. Rosser A. E., Bachoud-Levi A. C. Clinical trials of neural transplantation in Huntington's disease. Prog. Brain Res. 2012; 200:345-371. [PubMed]
36. Rossignol J., Fink K. D., Crane A. T., Davis K. K., Bombard M. C., Clerc S., Bavar A. M., Lowrance S. A., Song C., Witte S. Reductions in behavioral deficits and neuropathology in the R6/2 mouse model of Huntington's disease following transplantation of bone-marrow-derived mesenchymal stem cells is dependent on passage number. Stem Cell Res. Ther. 2015; 6:9. [PubMed]
37. Simmons D. A., Rex C. S., Palmer L., Pandyarajan V., Fedulov V., Gall C. M., Lynch G. Up-regulating BDNF with an ampakine rescues synaptic plasticity and memory in Huntington's disease knockin mice. Proc. Natl. Acad. Sci. USA. 2009; 106:4906-4911. [PubMed]
38. Spinelli K. J., Taylor J. K., Osterberg V. R., Churchill M. J., Pollock E., Moore C., Meshul C. K., Unni V. K. Presynaptic alpha-synuclein aggregation in a mouse model of Parkinson's disease. J. Neurosci. 2014; 34:2037-2050. [PubMed]
39. Strand A. D., Baguet Z. C., Aragaki A. K., Holmans P., Yang L., Cleren C., Beal M. F., Jones L., Kooperberg C., Olson J. M. Expression profiling of Huntington's disease models suggests that brain-derived neurotrophic factor depletion plays a major role in striatal degeneration. J. Neurosci. 2007; 27:11758-11768. [PubMed]
40. The Huntington's Disease Collaborative Research Group A novel gene containing a trinucleotide repeat that is expanded and unstable on Huntington's disease chromosomes. Cell. 1993; 72:971-983. [PubMed]
41. Vashishtha M., Ng C. W., Yildirim F., Gipson T. A., Kratter I. H., Bodai L., Song W., Lau A., Labadorf A., Vogel-Ciernia A. Targeting H3K4 trimethylation in Huntington disease. Proc. Natl. Acad. Sci. USA. 2013; 110:e3027-e3036. [PubMed]
42. Vonsattel J. P., DiFiglia M. Huntington disease. J. Neuropathol. Exp. Neurol. 1998; 57:369-384.[PubMed]
43. Walker R. H., Moore C., Davies G., Dirling L. B., Koch R. J., Meshul C. K. Effects of subthalamic nucleus lesions and stimulation upon corticostriatal afferents in the 6-hydroxydopamine-lesioned rat. PLoS One. 2012; 7:e32919. [PubMed]
44. Watson M. B., Richter F., Lee S. K., Gabby L., Wu J., Masliah E., Effros R. B., Chesselet M. F. Regionally-specific microglial activation in young mice over-expressing human wildtype alpha-synuclein. Exp. Neurol. 2012; 237:318-334. [PubMed]
45. Zuccato C., Marullo M., Vitali B., Tarditi A., Mariotti C., Valenza M., Lahiri N., Wild E. J., Sassone J., Ciammola A. Brain-derived neurotrophic factor in patients with Huntington's disease. PLoS One. 2011; 6:e22966

Sequence Listing:

NM_030752.2 and Homo sapiens t-complex 1 (TCP1), transcript
variant 1, mRNA
(SEQ ID NO.: 1)
GTCCTGTTTCTCTCCCTGTTGTCCCTGCCTCTTTTTCCTTCCCGCCGTGCCCCGCGG
CCGGGCCGGGGCAGCCGGGAAGCGGGTGGGGTGGTGTGTTACCCAGTAGCTCCT
GGGACATCGCTCGGGTACGCTCCACGCCGTCGCAGCCACTGCTGTGGTCGCCGGT
CGGCCGAGGGGCCGCGATACTGGTTGCCCGCGGTGTAAGCAGAATTCGACGTGT
ATCGCTGCCGTCAAGATGGAGGGGCCTTTGTCCGTGTTCGGTGACCGCAGCACTG
GGGAAACGATCCGCTCCCAAAACGTTATGGCTGCAGCTTCGATTGCCAATATTGT
AAAAAGTTCTCTTGGTCCAGTTGGCTTGGATAAAATGTTGGTGGATGATATTGGT
GATGTAACCATTACTAACGATGGTGCAACCATCCTGAAGTTACTGGAGGTAGAA
CATCCTGCAGCTAAAGTTCTTTGTGAGCTGGCTGATCTGCAAGACAAAGAAGTTG
GAGATGGAACTACTTCAGTGGTTATTATTGCAGCAGAACTCCTAAAAAATGCAG
ATGAATTAGTCAAACAGAAAATTCATCCCACATCAGTTATTAGTGGCTATCGACT
TGCTTGCAAGGAAGCAGTGCGTTATATCAATGAAAACCTAATTGTTAACACAGAT
GAACTGGGAAGAGATTGCCTGATTAATGCTGCTAAGACATCCATGTCTTCCAAAA
TCATTGGAATAAATGGTGATTTCTTTGCTAACATGGTAGTAGATGCTGTACTTGCT
ATTAAATACACAGACATAAGAGGCCAGCCACGCTATCCAGTCAACTCTGTTAATA
TTTTGAAAGCCCATGGGAGAAGTCAAATGGAGAGTATGCTCATCAGTGGCTATG
CACTCAACTGTGTGGTGGGATCCCAGGGCATGCCCAAGAGAATCGTAAATGCAA
AAATTGCTTGCCTTGACTTCAGCCTGCAAAAAACAAAAATGAAGCTTGGTGTACA
GGTGGTCATTACAGACCCTGAAAAACTGGACCAAATTAGACAGAGAGAATCAGA
TATCACCAAGGAGAGAATTCAGAAGATCCTGGCAACTGGTGCCAATGTTATTCTA
ACCACTGGTGGAATTGATGATATGTGTCTGAAGTATTTTGTGGAGGCTGGTGCTA
TGGCAGTTAGAAGAGTTTTAAAAAGGGACCTTAAACGCATTGCCAAAGCTTCTG
GAGCAACTATTCTGTCAACCCTGGCCAATTTGGAAGGTGAAGAAACTTTTGAAGC
TGCAATGTTGGGACAGGCAGAAGAAGTGGTACAGGAGAGAATTTGTGATGATGA
GCTGATCTTAATCAAAAATACTAAGGCTCGTACGTCTGCATCGATTATCTTACGT
GGGGCAAATGATTTCATGTGTGATGAGATGGAGCGCTCTTTACATGATGCACTTT
GTGTAGTGAAGAGAGTTTTGGAGTCAAAATCTGTGGTTCCCGGTGGGGGTGCTGT
AGAAGCAGCCCTTTCCATATACCTTGAAAACTATGCAACCAGCATGGGGTCTCGG
GAACAGCTTGCGATTGCAGAGTTTGCAAGATCACTTCTTGTTATTCCCAATACAC
TAGCAGTTAATGCTGCCCAGGACTCCACAGATCTGGTTGCAAAATTAAGAGCTTT
TCATAATGAGGCCCAGGTTAACCCAGAACGTAAAAATCTAAAATGGATTGGTCTT
GATTTGAGCAATGGTAAACCTCGAGACAACAAACAAGCAGGGGTGTTTGAACCA
ACCATAGTTAAAGTTAAGAGTTTGAAATTTGCAACAGAAGCTGCAATCACCATTC
TTCGAATTGATGATCTTATTAAATTACATCCAGAAAGTAAAGATGATAAACATGG
AAGTTATGAAGATGCTGTTCACTCTGGAGCCCTTAATGATTGATCTGATGTTCCTT
TTATTTATAACAATGTTAAATGCAATTGTCTTGTACCTTGAGTTGAGTATTACACA
TTAAAGTAAAGTACAAGCTGTAAACTTGGGTTTTTGTGATGTAGGAAATGGTTTC
CATCTGTACTTTGGTCCTCTGATTTCACATATTGCAACCTAGTACTTTATTAGTTT
AAAAAGAAATTGAGGTTGTTCAAAGTTTAAGCAATTCATTCTCTCTGAACACACA
TTGCTATTCCCATCCCACCCCCAATGCACAGGGCTGCAACACCACGACTTCTGCC
CATTCTCTCCAGTGTGTGTAACAGGGTCACAAGAATTCGACAGCCAGATGCTCCA
AGAGGGTGGCCCAAGGCTATAGCCCCTCCTTCAATATTGACCTAACGGGGGAGA
AAAGATTTAGATTGTTTATTCTTCTGTGGACACAGTTTAAAATCTTAAACTTGTCT
TTTTCCTCTTAATGTATCAGCATGCTACCCTTTCAAACTCAAATTTTCATTTTAACT
GCTTAGGAATAAATTTACACCTTTGTGAAAATTCAAAAAAAAAAA

| FEATURES | Location/Qualifiers |
|---|---|
| source | 1 . . . 2463 |
| | /organism = “Homo sapiens” |
| | /mol_type = “mRNA” |
| | /db_xref = “taxon: 9606” |
| | /chromosome = “6” |
| | /map = “6q25.3” |
| gene | 1 . . . 2463 |
| | /gene = “TCP1” |
| | /gene synonym = “CCT-alpha; CCT1; CCTa; D6S230E; |
| | TCP-1-alpha” |
| | /note = “t-complex 1” |
| | /db_xref = “GeneID: 6950” |
| | /db_xref = “HGNC:HGNC: 11655” |
| | /db_xref = “MIM: 186980” |
| exon | 1 . . . 299 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| misc_feature | 104 . . . 106 |
| | /gene = “TCP1” |
| | /gene_synonym = “CCT-alpha; CCT1; CCTa; D6S230E; |
| | TCP-1-alpha” |
| | /note = “upstream in-frame stop codon” |
| CDS | 236 . . . 1906 |
| | /gene = “TCP1” |
| | /gene_synonym = “CCT-alpha; CCT1; CCTa; D6S230E; |
| | TCP-1-alpha” |
| | /note = “isoform a is encoded by transcript variant 1; |
| | T-complex protein 1, alpha subunit; tailless complex |
| | polypeptide 1; T-complex protein 1 subunit alpha; |
| | t-complex 1 protein” |
| | /codon_start = 1 |
| | /product = “T-complex protein 1 subunit alpha isoform a” |
| | /protein_id = “NP_110379.2” |
| | /db_xref = “CCDS: CCDS5269.1” |
| | /db_xref = “GeneID: 6950” |
| | /db_xref = “HGNC:HGNC: 11655” |
| | /db_xref = “MIM: 186980” |
| | /translation = (SEQ ID NO.: 2) |

“MEGPLSVFGDRSTGETIRSQNVMAAASIANIVKSSLGPVGLDKMLVDDIGDVTITND
GATILKLLEVEHPAAKVLCELADLQDKEVGDGTTSVVIIAAELLKNADELVKQKIHP
TSVISGYRLACKEAVRYINENLIVNTDELGRDCLINAAKTSMSSKIIGINGDFFANMV
VDAVLAIKYTDIRGQPRYPVNSVNILKAHGRSQMESMLISGYALNCVVGSQGMPKRI
VNAKIACLDFSLQKTKMKLGVQVVITDPEKLDQIRQRESDITKERIQKILATGANVILT
TGGIDDMCLKYFVEAGAMAVRRVLKRDLKRIAKASGATILSTLANLEGEETFEAAM
LGQAEEVVQERICDDELILIKNTKARTSASIILRGANDFMCDEMERSLHDALCVVKRV
LESKSVVPGGGAVEAALSIYLENYATSMGSREQLAIAEFARSLLVIPNTLAVNAAQDS
TDLVAKLRAFHNEAQVNPERKNLKWIGLDLSNGKPRDNKQAGVFEPTIVKVKSLKF
ATEAAITILRIDDLIKLHPESKDDKHGSYEDAVHSGALND”

| | |
|---|---|
| misc_feature | 236 . . . 238 |
| | /gene = “TCP1” |
| | /gene_synonym = “CCT-alpha; CCT1; CCTa; D6S230E; |
| | TCP-1-alpha” |
| | /experiment = “experimental evidence, no additional details |
| | recorded” |
| | /note = “N-acetylmethionine. {ECO: 0000244\|PubMed: 19413330, |
| | ECO: 0000244\|PubMed: 22223895, ECO: 0000244\|PubMed: 22814378, |
| | ECO: 0000269\|PubMed: 12665801}; propagated from |
| | UniProtKB/Swiss-Prot (P17987.1); acetylation site” |
| misc_feature | 251 . . . 253 |
| | /gene = “TCP1” |
| | /gene_synonym = “CCT-alpha; CCT1; CCTa; D6S230E; |
| | TCP-1-alpha” |
| | /experiment = “experimental evidence, no additional details |
| | recorded” |
| | /note = “Phosphoserine. {ECO: 0000244\|PubMed: 23186163}; |
| | propagated from UniProtKB/Swiss-Prot (P17987.1); |
| | phosphorylation site” |
| misc_feature | 776 . . . 778 |
| | /gene = “TCP1” |
| | /gene_synonym = “CCT-alpha; CCT1; CCTa; D6S230E; |
| | TCP-1-alpha” |
| | /experiment = “experimental evidence, no additional details |
| | recorded” |
| | /note = “Phosphotyrosine. {ECO: 0000244\|PubMed: 19690332}; |
| | propagated from UniProtKB/Swiss-Prot (P17987.1); |
| | phosphorylation site” |
| misc_feature | 830 . . . 832 |
| | /gene = “TCP1” |
| | /gene_synonym = “CCT-alpha; CCT1; CCTa; D6S230E; |
| | TCP-1-alpha” |
| | /experiment = “experimental evidence, no additional details |
| | recorded” |
| | /note = “N6-acetyllysine. {ECO: 0000244\|PubMed: 19608861}; |
| | propagated from UniProtKB/Swiss-Prot (P17987.1); |
| | acetylation site” |
| misc_feature | 1433 . . . 1435 |
| | /gene = “TCP1” |
| | /gene_synonym = “CCT-alpha; CCT1; CCTa; D6S230E; |
| | TCP-1-alpha” |
| | /experiment = “experimental evidence, no additional details |
| | recorded” |
| | /note = “N6-acetyllysine. {ECO: 0000244\|PubMed: 19608861}; |
| | propagated from UniProtKB/Swiss-Prot (P17987.1); |
| | acetylation site” |
| misc_feature | 1706 . . . 1708 |
| | /gene = “TCP1” |
| | /gene_synonym = “CCT-alpha; CCT1; CCTa; D6S230E; |
| | TCP-1-alpha” |
| | /experiment = “experimental evidence, no additional details |
| | recorded” |
| | /note = “Phosphoserine. {ECO: 0000244\|PubMed: 23186163}; |
| | propagated from UniProtKB/Swiss-Prot (P17987.1); |
| | phosphorylation site” |
| misc_feature | 1715 . . . 1717 |
| | /gene = “TCP1” |
| | /gene_synonym = “CCT-alpha; CCT1; CCTa; D6S230E; |
| | TCP-1-alpha” |
| | /experiment = “experimental evidence, no additional details |
| | recorded” |
| | /note = “N6-acetyllysine. {ECO: 0000250\|UniProtKB: P11983}; |
| | propagated from UniProtKB/Swiss-Prot (P17987.1); |
| | acetylation site” |
| misc_feature | 1865 . . . 1867 |
| | /gene = “TCP1” |
| | /gene_synonym = “CCT-alpha; CCT1; CCTa; D6S230E; |
| | TCP-1-alpha” |
| | /experiment = “experimental evidence, no additional details |
| | recorded” |
| | /note = “Phosphoserine. {ECO: 0000244\|PubMed: 18669648, |
| | ECO: 0000244\|PubMed: 19690332, ECO: 0000244\|PubMed: 20068231, |
| | ECO: 0000244\|PubMed: 21406692, ECO: 0000244\|PubMed: 23186163, |
| | ECO: 0000244\|PubMed: 24275569}; propagated from |
| | UniProtKB/Swiss-Prot (P17987.1); phosphorylation site” |
| misc_feature | 1886 . . . 1888 |
| | /gene = “TCP1” |
| | /gene_synonym = “CCT-alpha; CCT1; CCTa; D6S230E; |
| | TCP-1-alpha” |
| | /experiment = “experimental evidence, no additional details |
| | recorded” |
| | /note = “Phosphoserine. {ECO: 0000244\|PubMed: 20068231, |
| | ECO: 0000244\|PubMed: 21406692, ECO: 0000244\|PubMed: 23186163}; |
| | propagated from UniProtKB/Swiss-Prot (P17987.1); |
| | phosphorylation site” |
| exon | 300 . . . 385 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 386 . . . 514 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 515 . . . 612 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 613 . . . 723 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 724 . . . 905 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 906 . . . 1032 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 1033 . . . 1208 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| STS | 1073 . . . 1300 |
| | /gene = “TCP1” |
| | /gene_synonym = “CCT-alpha; CCT1; CCTa; D6S230E; |
| | TCP-1-alpha” |
| | /standard_name = “GDB: 451649” |
| | /db_xref = “UniSTS: 157336” |
| exon | 1209 . . . 1332 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 1333 . . . 1525 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| STS | 1493 . . . 1674 |
| | /gene = “TCP1” |
| | /gene_synonym = “CCT-alpha; CCT1; CCTa; D6S230E; |
| | TCP-1-alpha” |
| | /standard_name = “G06897” |
| | /db_xref = “UniSTS: 35313” |
| exon | 1526 . . . 1689 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 1690 . . . 2453 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| STS | 1857 . . . 1964 |
| | /gene = “TCP1” |
| | /gene_synonym = “CCT-alpha; CCT1; CCTa; D6S230E; |
| | TCP-1-alpha” |
| | /standard_name = “SHGC-36020” |
| | /db_xref = “UniSTS: 22807” |
| regulatory | 1975 . . . 1980 |
| | /regulatory_class = “polyA_signal_sequence” |
| | /gene = “TCP1” |
| | /gene_synonym = “CCT-alpha; CCT1; CCTa; D6S230E; |
| | TCP-1-alpha” |
| polyA_site | 1999 |
| | /gene = “TCP1” |
| | /gene_synonym = “CCT-alpha; CCT1; CCTa; D6S230E; |
| | TCP-1-alpha” |
| | /experiment = “experimental evidence, no additional details |
| | recorded” |
| STS | 2009 . . . 2140 |
| | /gene = “TCP1” |
| | /gene_synonym = “CCT-alpha; CCT1; CCTa; D6S230E; |
| | TCP-1-alpha” |
| | /standard_name = “D6S1840” |
| | /db_xref = “UniSTS: 58762” |
| regulatory | 2426 . . . 2431 |
| | /regulatory_class = “polyA_signal_sequence” |
| | /gene = “TCP1” |
| | /gene_synonym = “CCT-alpha; CCT1; CCTa; D6S230E; |
| | TCP-1-alpha” |
| polyA_site | 2452 |
| | /gene = “TCP1” |
| | /gene_synonym = “CCT-alpha; CCT1; CCTa; D6S230E; |
| | TCP-1-alpha” |

NM_001008897.1 Homo sapiens t-complex 1 (TCP1), transcript
variant 2, mRNA
(SEQ ID NO:. 3)
GTCCTGTTTCTCTCCCTGTTGTCCCTGCCTCTTTTTCCTTCCCGCCGTGCCCCGCGG
CCGGGCCGGGGCAGCCGGGAAGCGGGTGGGGTGGTGTGTTACCCAGTAGCTCCT
GGGACATCGCTCGGGTACGCTCCACGCCGTCGCAGCCACTGCTGTGGTCGCCGGT
CGGCCGAGGGGCCGCGATACTGGTTGCCCGCGGTGTAAGCAGAATTCGACGTGT
ATCGCTGCCGTCAAGATGGAGGGGCCTTTGTCCGTGTTCGGTGACCGCAGCACTG
GGGAAACGATCCGCTCCCAAAACGGATGTAACCATTACTAACGATGGTGCAACC
ATCCTGAAGTTACTGGAGGTAGAACATCCTGCAGCTAAAGTTCTTTGTGAGCTGG
CTGATCTGCAAGACAAAGAAGTTGGAGATGGAACTACTTCAGTGGTTATTATTGC
AGCAGAACTCCTAAAAAATGCAGATGAATTAGTCAAACAGAAAATTCATCCCAC
ATCAGTTATTAGTGGCTATCGACTTGCTTGCAAGGAAGCAGTGCGTTATATCAAT
GAAAACCTAATTGTTAACACAGATGAACTGGGAAGAGATTGCCTGATTAATGCT
GCTAAGACATCCATGTCTTCCAAAATCATTGGAATAAATGGTGATTTCTTTGCTA
ACATGGTAGTAGATGCTGTACTTGCTATTAAATACACAGACATAAGAGGCCAGC
CACGCTATCCAGTCAACTCTGTTAATATTTTGAAAGCCCATGGGAGAAGTCAAAT
GGAGAGTATGCTCATCAGTGGCTATGCACTCAACTGTGTGGTGGGATCCCAGGGC
ATGCCCAAGAGAATCGTAAATGCAAAAATTGCTTGCCTTGACTTCAGCCTGCAAA
AAACAAAAATGAAGCTTGGTGTACAGGTGGTCATTACAGACCCTGAAAAACTGG
ACCAAATTAGACAGAGAGAATCAGATATCACCAAGGAGAGAATTCAGAAGATCC
TGGCAACTGGTGCCAATGTTATTCTAACCACTGGTGGAATTGATGATATGTGTCT
GAAGTATTTTGTGGAGGCTGGTGCTATGGCAGTTAGAAGAGTTTTAAAAAGGGA
CCTTAAACGCATTGCCAAAGCTTCTGGAGCAACTATTCTGTCAACCCTGGCCAAT
TTGGAAGGTGAAGAAACTTTTGAAGCTGCAATGTTGGGACAGGCAGAAGAAGTG
GTACAGGAGAGAATTTGTGATGATGAGCTGATCTTAATCAAAAATACTAAGGCT
CGTACGTCTGCATCGATTATCTTACGTGGGGCAAATGATTTCATGTGTGATGAGA
TGGAGCGCTCTTTACATGATGCACTTTGTGTAGTGAAGAGAGTTTTGGAGTCAAA
ATCTGTGGTTCCCGGTGGGGGTGCTGTAGAAGCAGCCCTTTCCATATACCTTGAA
AACTATGCAACCAGCATGGGGTCTCGGGAACAGCTTGCGATTGCAGAGTTTGCA
AGATCACTTCTTGTTATTCCCAATACACTAGCAGTTAATGCTGCCCAGGACTCCA
CAGATCTGGTTGCAAAATTAAGAGCTTTTCATAATGAGGCCCAGGTTAACCCAGA
ACGTAAAAATCTAAAATGGATTGGTCTTGATTTGAGCAATGGTAAACCTCGAGAC
AACAAACAAGCAGGGGTGTTTGAACCAACCATAGTTAAAGTTAAGAGTTTGAAA
TTTGCAACAGAAGCTGCAATCACCATTCTTCGAATTGATGATCTTATTAAATTAC
ATCCAGAAAGTAAAGATGATAAACATGGAAGTTATGAAGATGCTGTTCACTCTG
GAGCCCTTAATGATTGATCTGATGTTCCTTTTATTTATAACAATGTTAAATGCAAT
TGTCTTGTACCTTGAGTTGAGTATTACACATTAAAGTAAAGTACAAGCTGTAAAC
TTGGGTTTTTGTGATGTAGGAAATGGTTTCCATCTGTACTTTGGTCCTCTGATTTC
ACATATTGCAACCTAGTACTTTATTAGTTTAAAAAGAAATTGAGGTTGTTCAAAG
TTTAAGCAATTCATTCTCTCTGAACACACATTGCTATTCCCATCCCACCCCCAATG
CACAGGGCTGCAACACCACGACTTCTGCCCATTCTCTCCAGTGTGTGTAACAGGG
TCACAAGAATTCGACAGCCAGATGCTCCAAGAGGGTGGCCCAAGGCTATAGCCC
CTCCTTCAATATTGACCTAACGGGGGAGAAAAGATTTAGATTGTTTATTCTTCTGT
GGACACAGTTTAAAATCTTAAACTTGTCTTTTTCCTCTTAATGTATCAGCATGCTA
CCCTTTCAAACTCAAATTTTCATTTTAACTGCTTAGGAATAAATTTACACCTTTGT
GAAAATTCAAAAAAAAAAA

| FEATURES | Location/Qualifiers |
|---|---|
| source | 1 . . . 2377 |
| | /organism = “Homo sapiens” |
| | /mol_type = “mRNA” |
| | /db_xref = “taxon: 9606” |
| | /chromosome = “6” |
| | /map = “6q25.3” |
| gene | 1 . . . 2377 |
| | /gene = “TCP1” |
| | /gene_synonym = “CCT-alpha; CCT1; CCTa; D6S230E; |
| | TCP-1-alpha” |
| | /note = “t-complex 1” |
| | /db_xref = “GeneID: 6950” |
| | /db_xref = “HGNC:HGNC: 11655” |
| | /db_xref = “MIM: 186980” |
| exon | 1 . . . 299 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 300 . . . 428 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 429 . . . 526 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 527 . . . 637 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| CDS | 615 . . . 1820 |
| | /gene = “TCP1” |
| | /gene_synonym = “CCT-alpha; CCT1; CCTa; D6S230E; |
| | TCP-1-alpha” |
| | /note = “isoform b is encoded by transcript variant 2; |
| | T-complex protein 1, alpha subunit; tailless complex |
| | polypeptide 1; T-complex protein 1 subunit alpha; |
| | t-complex 1 protein” |
| | /codon_start = 1 |
| | /product = “T-complex protein 1 subunit alpha isoform b” |
| | /protein_id = “NP_001008897.1” |
| | /db_xref = “CCDS: CCDS43522.1” |
| | /db_xref = “GeneID: 6950” |
| | /db_xref = “HGNC:HGNC: 11655” |
| | /db_xref = “MIM: 186980” |
| | /translation = (SEQ ID NO:. 4) |

“MSSKIIGINGDFFANMVVDAVLAIKYTDIRGQPRYPVNSVNILKAHGRSQMESMLIS
GYALNCVVGSQGMPKRIVNAKIACLDFSLQKTKMKLGVQVVITDPEKLDQIRQRES
DITKERIQKILATGANVILTTGGIDDMCLKYFVEAGAMAVRRVLKRDLKRIAKASGA
TILSTLANLEGEETFEAAMLGQAEEVVQERICDDELILIKNTKARTSASIILRGANDFM
CDEMERSLHDALCVVKRVLESKSVVPGGGAVEAALSIYLENYATSMGSREQLAIAEF
ARSLLVIPNTLAVNAAQDSTDLVAKLRAFHNEAQVNPERKNLKWIGLDLSNGKPRD
NKQAGVFEPTIVKVKSLKFATEAAITILRIDDLIKLHPESKDDKHGSYEDAVHSGALN
D”

| | |
|---|---|
| exon | 638 . . . 819 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 820 . . . 946 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 947 . . . 1122 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| STS | 987 . . . 1214 |
| | /gene = “TCP1” |
| | /gene_synonym = “CCT-alpha; CCT1; CCTa; D6S230E; |
| | TCP-1-alpha” |
| | /standard_name = “GDB: 451649” |
| | /db_xref = “UniSTS: 157336” |
| exon | 1123 . . . 1246 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 1247 . . . 1439 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| STS | 1407 . . . 1588 |
| | /gene = “TCP1” |
| | /gene_synonym = “CCT-alpha; CCT1; CCTa; D6S230E; |
| | TCP-1-alpha” |
| | /standard_name = “G06897” |
| | /db_xref = “UniSTS: 35313” |
| exon | 1440 . . . 1603 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| exon | 1604 . . . 2367 |
| | /gene = “TCP1” |
| | /gene_synonym = “CCT-alpha; CCT1; CCTa; D6S230E; |
| | TCP-1-alpha” |
| | /inference = “alignment:Splign: 2.1.0” |
| STS | 1771 . . . 1878 |
| | /gene = “TCP1” |
| | /gene_synonym = “CCT-alpha; CCT1; CCTa; D6S230E; |
| | TCP-1-alpha” |
| | /standard_name = “SHGC-36020” |
| | /db_xref = “UniSTS: 22807” |
| regulatory | 1889 . . . 1894 |
| | /regulatory_class = “polyA_signal_sequence” |
| | /gene = “TCP1” |
| | /gene_synonym = “CCT-alpha; CCT1; CCTa; D6S230E; |
| | TCP-1-alpha” |
| polyA_site | 1913 |
| | /gene = “TCP1” |
| | /gene_synonym = “CCT-alpha; CCT1; CCTa; D6S230E; |
| | TCP-1-alpha” |
| STS | 1923 . . . 2054 |
| | /gene = “TCP1” |
| | /gene_synonym = “CCT-alpha; CCT1; CCTa; D6S230E; |
| | TCP-1-alpha” |
| | /standard_name = “D6S1840” |
| | /db_xref = “UniSTS: 58762” |
| regulatory | 2340 . . . 2345 |
| | /regulatory_class = “polyA_signal_sequence” |
| | /gene = “TCP1” |
| | /gene_synonym = “CCT-alpha; CCT1; CCTa; D6S230E; |
| | TCP-1-alpha” |
| polyA_site | 2366 |
| | /gene = “TCP1” |
| | /gene_synonym = “CCT-alpha; CCT1; CCTa; D6S230E; |
| | TCP-1-alpha” |

NM_001143805.1 Homo sapiens brain derived neurotrophic factor
(BDNF), transcript variant 7, mRNA
(SEQ ID NO: 5)
AATATCAAGTATCACTTAATTAGAGATTTTTAAGCCTTTTCCTCCTGCTGTGCCGG
GTGTGTAATCCGGGCGATAGGAGTCCATTCAGCACCTTGGACAGAGCCAACGGA
TTTGTCCGAGGTGGCGGTACCCCCAGTTCCACCAGGTGAGAAGAGTGATGACCAT
CCTTTTCCTTACTATGGTTATTTCATACTTTGGTTGCATGAAGGCTGCCCCCATGA
AAGAAGCAAACATCCGAGGACAAGGTGGCTTGGCCTACCCAGGTGTGCGGACCC
ATGGGACTCTGGAGAGCGTGAATGGGCCCAAGGCAGGTTCAAGAGGCTTGACAT
CATTGGCTGACACTTTCGAACACGTGATAGAAGAGCTGTTGGATGAGGACCAGA
AAGTTCGGCCCAATGAAGAAAACAATAAGGACGCAGACTTGTACACGTCCAGGG
TGATGCTCAGTAGTCAAGTGCCTTTGGAGCCTCCTCTTCTCTTTCTGCTGGAGGAA
TACAAAAATTACCTAGATGCTGCAAACATGTCCATGAGGGTCCGGCGCCACTCTG
ACCCTGCCCGCCGAGGGGAGCTGAGCGTGTGTGACAGTATTAGTGAGTGGGTAA
CGGCGGCAGACAAAAAGACTGCAGTGGACATGTCGGGCGGGACGGTCACAGTCC
TTGAAAAGGTCCCTGTATCAAAAGGCCAACTGAAGCAATACTTCTACGAGACCA
AGTGCAATCCCATGGGTTACACAAAAGAAGGCTGCAGGGGCATAGACAAAAGGC
ATTGGAACTCCCAGTGCCGAACTACCCAGTCGTACGTGCGGGCCCTTACCATGGA
TAGCAAAAAGAGAATTGGCTGGCGATTCATAAGGATAGACACTTCTTGTGTATGT
ACATTGACCATTAAAAGGGGAAGATAGTGGATTTATGTTGTATAGATTAGATTAT
ATTGAGACAAAAATTATCTATTTGTATATATACATAACAGGGTAAATTATTCAGT
TAAGAAAAAAATAATTTTATGAACTGCATGTATAAATGAAGTTTATACAGTACAG
TGGTTCTACAATCTATTTATTGGACATGTCCATGACCAGAAGGGAAACAGTCATT
TGCGCACAACTTAAAAAGTCTGCATTACATTCCTTGATAATGTTGTGGTTTGTTGC
CGTTGCCAAGAACTGAAAACATAAAAAGTTAAAAAAAATAATAAATTGCATGCT
GCTTTAATTGTGAATTGATAATAAACTGTCCTCTTTCAGAAAACAGAAAAAAACA
CACACACACACAACAAAAATTTGAACCAAAACATTCCGTTTACATTTTAGACAGT
AAGTATCTTCGTTCTTGTTAGTACTATATCTGTTTTACTGCTTTTAACTTCTGATAG
CGTTGGAATTAAAACAATGTCAAGGTGCTGTTGTCATTGCTTTACTGGCTTAGGG
GATGGGGGATGGGGGGTATATTTTTGTTTGTTTTGTGTTTTTTTTTCGTTTGTTTGT
TTTGTTTTTTAGTTCCCACAGGGAGTAGAGATGGGGAAAGAATTCCTACAATATA
TATTCTGGCTGATAAAAGATACATTTGTATGTTGTGAAGATGTTTGCAATATCGA
TCAGATGACTAGAAAGTGAATAAAAATTAAGGCAACTGAACAAAAAAATGCTCA
CACTCCACATCCCGTGATGCACCTCCCAGGCCCCGCTCATTCTTTGGGCGTTGGT
CAGAGTAAGCTGCTTTTGACGGAAGGACCTATGTTTGCTCAGAACACATTCTTTC
CCCCCCTCCCCCTCTGGTCTCCTCTTTGTTTTGTTTTAAGGAAGAAAAATCAGTTG
CGCGTTCTGAAATATTTTACCACTGCTGTGAACAAGTGAACACATTGTGTCACAT
CATGACACTCGTATAAGCATGGAGAACAGTGATTTTTTTTTAGAACAGAAAACAA
CAAAAAATAACCCCAAAATGAAGATTATTTTTTATGAGGAGTGAACATTTGGGTA
AATCATGGCTAAGCTTAAAAAAAACTCATGGTGAGGCTTAACAATGTCTTGTAAG
CAAAAGGTAGAGCCCTGTATCAACCCAGAAACACCTAGATCAGAACAGGAATCC
ACATTGCCAGTGACATGAGACTGAACAGCCAAATGGAGGCTATGTGGAGTTGGC
ATTGCATTTACCGGCAGTGCGGGAGGAATTTCTGAGTGGCCATCCCAAGGTCTAG
GTGGAGGTGGGGCATGGTATTTGAGACATTCCAAAACGAAGGCCTCTGAAGGAC
CCTTCAGAGGTGGCTCTGGAATGACATGTGTCAAGCTGCTTGGACCTCGTGCTTT
AAGTGCCTACATTATCTAACTGTGCTCAAGAGGTTCTCGACTGGAGGACCACACT
CAAGCCGACTTATGCCCACCATCCCACCTCTGGATAATTTTGCATAAAATTGGAT
TAGCCTGGAGCAGGTTGGGAGCCAAATGTGGCATTTGTGATCATGAGATTGATGC
AATGAGATAGAAGATGTTTGCTACCTGAACACTTATTGCTTTGAAACTAGACTTG
AGGAAACCAGGGTTTATCTTTTGAGAACTTTTGGTAAGGGAAAAGGGAACAGGA
AAAGAAACCCCAAACTCAGGCCGAATGATCAAGGGGACCCATAGGAAATCTTGT
CCAGAGACAAGACTTCGGGAAGGTGTCTGGACATTCAGAACACCAAGACTTGAA
GGTGCCTTGCTCAATGGAAGAGGCCAGGACAGAGCTGACAAAATTTTGCTCCCC
AGTGAAGGCCACAGCAACCTTCTGCCCATCCTGTCTGTTCATGGAGAGGGTCCCT
GCCTCACCTCTGCCATTTTGGGTTAGGAGAAGTCAAGTTGGGAGCCTGAAATAGT
GGTTCTTGGAAAAATGGATCCCCAGTGAAAACTAGAGCTCTAAGCCCATTCAGCC
CATTTCACACCTGAAAATGTTAGTGATCACCACTTGGACCAGCATCCTTAAGTAT
CAGAAAGCCCCAAGCAATTGCTGCATCTTAGTAGGGTGAGGGATAAGCAAAAGA
GGATGTTCACCATAACCCAGGAATGAAGATACCATCAGCAAAGAATTTCAATTT
GTTCAGTCTTTCATTTAGAGCTAGTCTTTCACAGTACCATCTGAATACCTCTTTGA
AAGAAGGAAGACTTTACGTAGTGTAGATTTGTTTTGTGTTGTTTGAAAATATTAT
CTTTGTAATTATTTTTAATATGTAAGGAATGCTTGGAATATCTGCTATATGTCAAC
TTTATGCAGCTTCCTTTTGAGGGACAAATTTAAAACAAACAACCCCCCATCACAA
ACTTAAAGGATTGCAAGGGCCAGATCTGTTAAGTGGTTTCATAGGAGACACATCC
AGCAATTGTGTGGTCAGTGGCTCTTTTACCCAATAAGATACATCACAGTCACATG
CTTGATGGTTTATGTTGACCTAAGATTTATTTTGTTAAAATCTCTCTCTGTTGTGTT
CGTTCTTGTTCTGTTTTGTTTTGTTTTTTAAAGTCTTGCTGTGGTCTCTTTGTGGCA
GAAGTGTTTCATGCATGGCAGCAGGCCTGTTGCTTTTTTATGGCGATTCCCATTGA
AAATGTAAGTAAATGTCTGTGGCCTTGTTCTCTCTATGGTAAAGATATTATTCACC
ATGTAAAACAAAAAACAATATTTATTGTATTTTAGTATATTTATATAATTATGTTA
TTGAAAAAAATTGGCATTAAAACTTAACCGCATCAGAACCTATTGTAAATACAA
GTTCTATTTAAGTGTACTAATTAACATATAATATATGTTTTAAATATAGAATTTTT
AATGTTTTTAAATATATTTTCAAAGTACATAAAA

| FEATURES | Location/Qualifiers |
|---|---|
| source | 1 . . . 3827 |
| | /organism = “Homo sapiens” |
| | /mol_type = “mRNA” |
| | /db_xref = “taxon: 9606” |
| | /chromosome = “11” |
| | /map = “11p14.1” |
| gene | 1 . . . 3827 |
| | /gene = “BDNF” |
| | /gene_synonym = “ANON2; BULN2” |
| | /note = “brain derived neurotrophic factor” |
| | /db_xref = “GeneID: 627” |
| | /db_xref = “HGNC:HGNC: 1033” |
| | /db_xref = “MIM: 113505” |
| exon | 1 . . . 136 |
| | /gene = “BDNF” |
| | /gene_synonym = “ANON2; BULN2” |
| | /inference = “alignment:Splign: 2.1.0” |
| misc_feature | 11 |
| | /gene = “BDNF” |
| | /gene_synonym = “ANON2; BULN2” |
| | /note = “alternative transcription initiation start site” |
| misc_feature | 12 |
| | /gene = “BDNF” |
| | /gene_synonym = “ANON2; BULN2” |
| | /note = “alternative transcription initiation start site” |
| misc_feature | 18 |
| | /gene = “BDNF” |
| | /gene_synonym = “ANON2; BULN2” |
| | /note = “alternative transcription initiation start site” |
| misc_feature | 27 |
| | /gene = “BDNF” |
| | /gene_synonym = “ANON2; BULN2” |
| | /note = “alternative transcription initiation start site” |
| misc_feature | 34 |
| | /gene = “BDNF” |
| | /gene_synonym = “ANON2; BULN2” |
| | /note = “alternative transcription initiation start site” |
| misc_feature | 74 . . . 76 |
| | /gene = “BDNF” |
| | /gene_synonym = “ANON2; BULN2” |
| | /note = “upstream in-frame stop codon” |
| exon | 137 . . . 3827 |
| | /gene = “BDNF” |
| | /gene_synonym = “ANON2; BULN2” |
| | /inference = “alignment:Splign: 2.1.0” |
| CDS | 158 . . . 901 |
| | /gene = “BDNF” |
| | /gene_synonym = “ANON2; BULN2” |
| | /note = “isoform a preproprotein is encoded by transcript |
| | variant 7; neurotrophin; abrineurin” |
| | /codon_start = 1 |
| | /product = “brain-derived neurotrophic factor isoform a |
| | preproprotein” |
| | /protein_id = “NP_001137277.1” |
| | /db_xref = “CCDS: CCDS7866.1” |
| | /db_xref = “GeneID: 627” |
| | /db_xref = “HGNC:HGNC: 1033” |
| | /db_xref = “MIM: 113505” |
| | /translation = (SEQ ID NO.: 6) |

“MTILFLTMVISYFGCMKAAPMKEANIRGQGGLAYPGVRTHGTLESVNGPKAGSRGL
TSLADTFEHVIEELLDEDQKVRPNEENNKDADLYTSRVMLSSQVPLEPPLLFLLEEYK
NYLDAANMSMRVRRHSDPARRGELSVCDSISEWVTAADKKTAVDMSGGTVTVLEK
VPVSKGQLKQYFYETKCNPMGYTKEGCRGIDKRHWNSQCRTTQSYVRALTMDSKK
RIGWRFIRIDTSCVCTLTIKRGR”

| | |
|---|---|
| sig_peptide | 158 . . . 211 |
| | /gene = “BDNF” |
| | /gene_synonym = “ANON2; BULN2” |
| | /inference = “COORDINATES: ab initio prediction: SignalP: 4.0” |
| misc_feature | 326 . . . 331 |
| | /gene = “BDNF” |
| | /gene_synonym = “ANON2; BULN2” |
| | /experiment = “experimental evidence, no additional details |
| | recorded” |
| | /note = “Cleavage, by S1P; propagated from |
| | UniProtKB/Swiss-Prot (P23560.1); cleavage site” |
| mat_peptide | 542 . . . 898 |
| | /gene = “BDNF” |
| | /gene_synonym = “ANON2; BULN2” |
| | /product = “Brain-derived neurotrophic factor” |
| | /experiment = “experimental evidence, no additional details |
| | recorded” |
| | /note = “propagated from UniProtKB/Swiss-Prot (P23560.1)” |
| STS | 163 . . . 771 |
| | /gene = “BDNF” |
| | /gene_synonym = “ANON2; BULN2” |
| | /standard_name = “BDNF” |
| | /db_xref = “UniSTS: 266531” |
| STS | 514 . . . 796 |
| | /gene = “BDNF” |
| | /gene_synonym = “ANON2; BULN2” |
| | /standard_name = “BDNF-1” |
| | /db_xref = “UniSTS: 253960” |
| STS | 578 . . . 1460 |
| | /gene = “BDNF” |
| | /gene_synonym = “ANON2; BULN2” |
| | /standard_name = “BDNF_2411” |
| | /db_xref = “UniSTS: 280459” |
| STS | 1062 . . . 1163 |
| | /gene = “BDNF” |
| | /gene_synonym = “ANON2; BULN2” |
| | /standard_name = “D11S4429” |
| | /db_xref = “UniSTS: 43225” |
| polyA_site | 3827 |
| | /gene = “BDNF” |
| | /gene_synonym = “ANON2; BULN2” |

ApiCCT1 (SEQ ID NO: 7):
MVPGYALNCTVASQAMPKRIAGGNVKIACLDLNLQKARMAMGVQINIDDPEQLEQI
RKREAGIVLERVKKIIDAGAQWLTIKGIDDLCLKEFVEAK1MGVRRCKKEDLRRIARA
TGATLVSSMSNLEGEETFESSYLGLCDEWQAKFSDDECILIKGTSKAAAAALE.
sApiCCT1 mRNA (SEQ ID NO: 8)
ATGTACAGGATGCAACTCCTGTCTTGCATTGCACTAAGTCTTGCACTTGTCACGA
ATTCTATCAGTGGCTATGCACTCAACTGTGTGGTGGGATCCCAGGGCATGCCCAA
GAGAATCGTAAATGCAAAAATTGCTTGCCTTGACTTCAGCCTGCAAAAAACAAA
AATGAAGCTTGGTGTACAGGTGGTCATTACAGACCCTGAAAAACTGGACCAAAT
TAGACAGAGAGAATCAGATATCACCAAGGAGAGAATTCAGAAGATCCTGGCAAC
TGGTGCCAATGTTATTCTAACCACTGGTGGAATTGATGATATGTGTCTGAAGTAT
TTTGTGGAGGCTGGTGCTATGGCAGTTAGAAGAGTTTTAAAAAGGGACCTTAAAC
GCATTGCCAAAGCTTCTGGAGCAACTATTCTGTCAACCCTGGCCAATTTGGAAGG
TGAAGAAACTTTTGAAGCTGCAATGTTGGGACAGGCAGAAGAAGTGGTACAGGA
GAGAATTTGTGATGATGAGCTGATCTTAATCAAAAATACTAAGGCTGCTGCGGCT
GCGGGTGGACACTACCCTTACGACGTGCCTGACTACGCCTGA
sApiCCT1 (SEQ ID NO: 9)
MYRMQLLSCIALSLALVTNSISGYALNCVVGSQGMPKRIVNAKIACLDFSLQKTKMK
LGVQVVITDPEKLDQIRQRESDITKERIQKILATGANVILTTGGIDDMCLKYFVEAGA
MAVRRVLKRDLKRIAKASGATILSTLANLEGEETFEAAMLGQAEEVVQERICDDELI
LIKNTKAAAAAGGHYPYDVPDYA

This listing of claims will replace all prior versions and listings of claims in the application:

1. A method to prepare a human neuronal stem cell (hNSC) from a human embryonic stem cell (hESC), the method comprising the steps of:
a) isolating at least one stem cell rosette from a population of embryoid bodies (EB) cultured in differentiation medium;
b) culturing at least one individual cell isolated from the rosette of step a) for an amount of time and under until conditions that provide for the generation of at least one rosette;
c) isolating an individual cell from the rosette of step b) into individual cells; and
d) culturing the at least one individual cell isolated from step c) for an amount of time and under until conditions that provide for the generation of confluent population of hNSCs.

2. The method of claim 1, further comprising one or more of:
wherein the isolation of the at least one individual cell from the rosette is performed manually;
wherein the isolation of the at least one individual cell from the rosette is performed enzymatically;
wherein the isolation of the at least one individual cell from the rosette of step a) is performed manually;
wherein the isolation of the at least one individual cell from the rosette of step a) is performed enzymatically;
wherein one or more of steps a) through c) is performed 2 or more times;
wherein at least one of steps a) through d) is performed manually;
wherein at least one of steps a) through d) is performed mechanically;
wherein the isolation of the rosette is performed digitally; or
wherein the at least one individual cell isolated in step c) is cultured for an effective amount of time on an ornithin/laminin coated plate in N2 medium to generate a confluent cell population of hNSCs.

3-9. (canceled)

10. The method of claim 1, further comprising one or more of the following: generating the embryoid bodies from ESI-017; culturing the embryoid body (EB) on an ultra-low attachment surface in EB medium; or genetically modifying the cell.

11. (canceled)

12. The method of claim 10, further comprising substituting N2 medium for the EB medium after the EBs have been cultured for an effective amount of time further to step a) on an ornithine/laminin coated surface.

13. The method of claim 12, further comprising substituting N2 medium for the EB medium after the EB have been cultured in the EB medium for an amount of time effective to produce at least one EB of step a).

14. (canceled)

15. The method of claim 2, further comprising culturing the confluent population of hNSCs with an effective amount of N2 medium.

16. The method of claim 15, further comprising expanding the population of cells.

17. (canceled)

18. The method of claim 10, wherein the cell is genetically modified by insertion of a transgene, or by modification by CRISPR.

19. The method of claim 18, wherein the transgene is ApiCCT1, a fragment thereof, or an equivalent of each thereof, and optionally wherein the transgene is overexpressed in the cell.

20. An hNSC prepared by the method of claim 15, and optionally wherein the cell expresses BNDF.

21. An hNSC prepared by the method of claim 10, wherein the hNSC expresses BNDF upon differentiation of the cell.

22. The hNSC of claim 21, wherein the cell is genetically modified by insertion of a transgene, or by CRISPR.

23. A population of cells of claim 20.

24. A composition comprising the isolated cell of claim 20 or a population thereof and a carrier.

25. (canceled)

26. The composition of claim 24 or 25, further comprising one or both of: preservative or cryoprotectant.

27. A method to deliver a transgene to a subject, or to genetically edit a cell in a subject in need thereof, comprising administering an effective amount of a cell of claim 20.

28-29. (canceled)

30. A method of treating a neurodegenerative disorder or enhancing synaptic connections in a subject in need thereof, comprising administering to the subject an effective amount of the isolated cell of claim 20.

31-33. (canceled)

34. A kit comprising an hESC and instructions to perform the method of claim 1.

35. A kit comprising the hNSC of claim 20, and instructions of use.

36. A non-human animal having the hNSC of claim 20 transplanted into the animal.

37. (canceled)
